(19) United States
(12) Patent Application Publication
(10) Pub. No.: US 2023/0076173 A1
(43) Pub. Date: Mar. 9, 2023

(54) ANTIMICROBIAL MATRIX AND USES THEREOF FOR ELIMINATING MICROORGANISMS

(71) Applicant: Yissum Research Development Company of the Hebrew University of Jerusalem Ltd., Jerusalem (IL)

(72) Inventor: Zvi HAYOUKA, Mazkeret Batya (IL)

(73) Assignee: Yissum Research Development Company of the Hebrew University of Jerusalem Ltd., Jerusalem (IL)

(21) Appl. No.: 17/760,357

(22) PCT Filed: Feb. 9, 2021
(86) PCT No.: PCT/IL2021/050152

§ 371 Date: Aug. 8, 2022

Related U.S. Application Data
(60) Provisional application No. 62/971,986, filed on Feb. 9, 2020.

(51) Int. Cl. A61L 2/232; C07K 17/06; C07K 17/08

(52) U.S. Cl. CPC A61L 2/232; C07K 17/06; C07K 17/08

(57) ABSTRACT

The present invention provides antimicrobial matrices comprising a water-insoluble polymer and a mixture comprising a plurality of synthetic peptides attached thereto via a linker, the peptides comprise cationic amino acid residues, hydrophobic amino acid residues, or combinations thereof, in random sequences. The invention further provides uses of the antimicrobial matrices for eliminating microorganisms, particularly pathogenic bacteria, from liquid or semi solid media including edible products or beverages.

### FIELD OF THE INVENTION

The present invention relates to antimicrobial matrices comprising a water-insoluble polymer and a mixture comprising a plurality of synthetic peptides attached thereto, the peptides comprising cationic amino acid residues and/or hydrophobic amino acid residues being organized in random sequences, and to uses thereof for eliminating microorganisms, particularly pathogenic bacteria, from various liquids or samples.

### BACKGROUND OF THE INVENTION

The presence of bacterial pathogens in potable water and food is a well-recognized cause of severe illnesses in humans and animals. Among bacterial pathogens, gram positive bacteria are wildly spread in samples like blood, plant material, water, soil, and feces. A whole series of pathogen germs, e.g., of the species Bacillus, Listeria, Clostridium, Staphylococcus, Streptococcus, Escherichia, Enterococcus, Micrococcus or mycobacteria, is particularly relevant in the food sector.

The group Bacillus cereus represents microorganisms of major economic and medical importance. The bacteria are closely related to each other within the group; a large amount of which are sequenced. They are wildly spread in nature and present in different kinds of food, mostly of plant origin. They are aerobic living, moving, rod shaped gram-positive bacteria. Due to their resistant endospores, they are able to survive different methods used to cure food, e.g., drying or heating. Frequently contaminated food is mainly starch containing food, cereals, rice, spices, vegetables and ready-to-eat products. Meat can be contaminated by using contaminated spices. Milk products are frequently contaminated because the spores survive pasteurization.

Listeria are human and animal pathogen bacteria, frequently present in food, particularly in fish, meat and milk products. The genus Listeria comprises six different species with 16 different serotypes. Although only a small portion of the food related diseases is caused by Listeria (about 1% in USA), almost 30% of the annually fatal diseases, caused by food pathogens, are caused by this germ. Affected are mainly immune suppressed human subjects, e.g., older subjects, diabetic patients, cancer patients and/or AIDS patients. Pregnant women and fetuses represent 25% of all cases of listeriosis patients.

Staphylococcus and Enterococcus are currently the most problematic pathogens associated with infectious diseases. These pathogens increasingly develop multi resistant germs (e.g. MRSA—multi resistant Staphylococcus aureus and VRE—vancomycin resistant Enterococcus) which lead to bad disease prognosis as well as to a dramatic increase of the costs of health care.

Microbial contamination, even if occurs at low concentrations, can put the consumers at risk, and therefore there is an ever-present need for removal or capture of the pathogens in both agricultural specimens (such as water or food products) and environmental specimens (such as surfaces in food processing plants).

Various methods have been used to separate microorganisms from specimens in which they reside. For example, antibodies coupled to magnetic beads were used to separate specific organisms from human fluids, food, and water. Also, cell surface derived lectins and carbohydrates have been proposed.

Haynie et al. (Antimicrob. Agents Chemother. 39: 301-307, 1995) discloses the preparation of certain synthetic peptides containing leucine and lysine or leucine, lysine and glycine immobilized on polyamide resin which showed antimicrobial activity against several bacteria.

Stratman et al. (Applied and Environ. Microbiol. 72: 5150-5158, 2006) discloses the use of the peptide aMptD immobilized on paramagnetic beads to capture Mycobacterium avian subsp. paratuberculosis in contaminated milk.

U.S. Pat. No. 8,710,179 discloses methods for concentrating microorganisms in a liquid sample or depleting microorganisms therefrom utilizing polymeric compounds having affinity to microbial cells. According to U.S. Pat. No. 8,710,179, the polymeric compounds are composed of a plurality of positively charged amino acid residues and two or more hydrophobic moieties, particularly the polymeric compounds are capryl- and/or lauryl-lysine conjugates.

U.S. Pat. No. 9,175,036 discloses antimicrobial water treatment membranes comprising a water treatment membrane covalently attached to one or more antimicrobial peptides or derivatives thereof, either directly or via one or more tether molecules. According to U.S. Pat. No. 9,175,036, the peptides are all-(D)-amino acid analogs of natural peptides or N-methylated analogs of natural peptides.

Hayouka et al. (J. Am. Chem. Soc. 135: 11748-11751, 2013) discloses peptide mixtures containing one type of a hydrophobic amino acid residue and one type of a cationic amino acid residue. The mixture was random in terms of sequence but highly controlled in terms of length. The antibacterial activity was shown to be selective for heterochiral binary mixtures but not for homochiral binary mixtures.

Cheriker et al. (Chem. Commun. 56: 11022-11025, 2020) discloses the antimicrobial activity of randomly-sequenced peptide mixtures bearing hydrophobic and cationic residues which were immobilized on beads and showed the high and broad bactericidal activity of these beads against various pathogenic bacteria.

WO 2017/134661 to the inventor of the present invention discloses random-sequence synthetic peptide mixtures for use in disrupting biofilms and/or preventing the formation of biofilms.

There is still an unmet need for improved means and methods which efficiently eliminate or exterminate pathogenic microorganisms in water and food systems.

### SUMMARY OF THE INVENTION

The present invention provides antimicrobial matrices comprising a water-insoluble polymer and an immobilized mixture comprising a plurality of synthetic peptides linked thereto via a linker, the mixture of the plurality of peptides comprises cationic amino acid residues and hydrophobic amino acid residues, wherein the cationic amino acid residues and the hydrophobic amino acid residues are organized in the peptides in random sequences. The present invention further provides an article adapted to eliminate or exterminate viable microorganisms from a liquid medium, the article containing the antimicrobial matrices of the present invention. The present invention further provides methods for eliminating or exterminating microorganisms from a liquid medium, the methods comprising contacting a liquid medium with the antimicrobial matrix of the present invention thereby eliminating or exterminating the microorganisms. In particular embodiments, the article and methods are intended for use with edible products or beverages.

The present invention is based in part on the unexpected findings that a matrix comprising a water-insoluble synthetic polymer and a mixture of a plurality of random sequence 20-mer peptides consisting of leucine and lysine residues attached to the water-insoluble polymer via a linker exerted pronounced antimicrobial activity. The antimicrobial activity of the matrix was similar to the antimicrobial activity of a soluble, non-attached random-sequence 20-mer peptide mixture consisting of leucine and lysine residues. Advantageously, the immobilized peptides linked to the insoluble matrix can be re-used in multiple cycles of decontamination of the liquid medium.

The present invention discloses that not only did mixtures of 20-mer random sequence peptides consisting of leucine and lysine exhibit antimicrobial activity while being attached via a linker to a water-insoluble polymer, but also mixtures of 10-mer random sequence peptides consisting of the same amino acid residues attached to the same polymer were capable of eliminating bacterial cells.

The present invention further discloses that phenylalanine or tryptophan as hydrophobic amino acid residues together with lysine endow the immobilized random sequence peptides with pronounced antimicrobial activity. However, the binary combination of leucine and lysine in the immobilized random sequence peptides was found to exhibit even higher antimicrobial activity.

Surprisingly, the ability of the immobilized random sequence peptides to eliminate viable bacterial cells from liquids, e.g., water or natural fruit juice, was observed after 2, 3, or even after 10 cycles of incubations with bacterial cells, followed by washes with various polar solvents and water. Not only did the immobilized peptides exhibit antimicrobial activity, but the antimicrobial activity was improved as a function of the number of recycles.

It is further disclosed that in order to exert antimicrobial activity, the random-sequence peptides should be attached or immobilized to the water-insoluble polymer via a linker which forms a distance between the peptide and the polymer of about 5 Å to about 20 Å. Without being bound to any theory or mechanism of action, it is suggested that a linker shorter than about 5 Å, e.g., amino methyl, does not endow the peptides sufficient conformational flexibility to interact with bacterial cells, and therefore such immobilized peptides exert very low or even undetectable antibacterial activity. A linker longer than about 20 Å endows the random-sequence peptides with high conformational flexibility such that the peptides interact with the water-insoluble polymer, rather than with bacterial cells, and therefore the antibacterial activity is significantly affected.

The present invention further discloses the unexpected findings that while a molar ratio between the total amount of one species of a cationic amino acid residue and the total amount of one species of a hydrophobic amino acid residue of the immobilized random-sequence peptides within the mixture can range from 10:1 to 1:10, respectively, a ratio of 3:1 to 1:1, or even 7:3, between the total amount of the cationic amino acid residue and the hydrophobic amino acid residue, respectively, enabled the immobilized random-sequence peptides to exert maximal antimicrobial activity, e.g., antibacterial activity. In contrast, a ratio of 1:1 between the total amount of one species of a cationic amino acid residue and the total amount of one species of a hydrophobic amino acid residue, respectively, within a mixture of soluble random-sequence peptides enabled the soluble random-sequence peptides to exert maximal antimicrobial activity, e.g., antibacterial activity, whereas a ratio of 7:3 between the total amount of the cationic amino acid residue and the hydrophobic amino acid residue, respectively, significantly reduced the antimicrobial activity, e.g. antibacterial activity, of the soluble random-sequence peptides. Without being bound to any theory or mechanism of action, it is speculated that due to the hydrophobicity of the water-insoluble polymer which the random-sequence peptides are attached to, a higher amount of cationic amino acid residues than hydrophobic amino acid residues is required in order to confer sufficient charge to the random-sequence peptides and thereby to achieve maximal antimicrobial activity of the mixture of the immobilized random-sequence peptides.

The mixture of the immobilized random-sequence peptides was shown in the present invention to have a broad antimicrobial activity such that gram-positive bacteria, gram-negative bacteria, and antibiotic-resistant bacteria were eradicated by the immobilized peptides. Due to their fast and effective antibacterial activity, the antimicrobial matrix of the present invention is useful for eradicating bacteria from water and food products, such as from drinking water or fruit juices, thus avoiding the need of thermal pasteurization. As thermal pasteurization is particularly undesired for fruit or vegetable juices which acquire different taste and appearance due to the heat treatment, the antimicrobial matrix of the present invention is therefore highly advantageous as a means of non-thermal pasteurization.

It is further disclosed that the amount of the peptides detached from the matrix was essentially negligible, reaching in some embodiments to a maximal amount of up to 0.05% of the bound peptides after numerous hours of incubation at 37° C. In additional embodiments, the maximal amount of the peptides detached from the matrix reached an amount of up to 0.02% of the bound peptide after numerous hours of incubation at 37° C.

Thus, the antimicrobial matrices of the present invention are highly efficient in eliminating or exterminating viable microorganisms, particularly pathogenic bacteria, from various samples. The matrices are shown to be re-usable and as such are cost effective.

Without being bound to any theory or mechanism of action, it is assumed that the peptides adsorb to the membrane of the bacterial cells, disrupt the membrane of the bacterial cells, and thereby kill the microorganisms.

According to one aspect, the present invention provides an antimicrobial matrix comprising a water-insoluble polymer, a linker, and a mixture comprising a plurality of synthetic peptides attached to the water-insoluble polymer via the linker,
wherein the synthetic peptides comprise cationic amino acid residues, hydrophobic amino acid residues, or any combinations thereof,
wherein the ratio between the total amount of the cationic amino acid residues and the total amount of the hydrophobic amino acid residues ranges between about 10:1 to about 1:10, and
wherein the cationic amino acid residues and the hydrophobic amino acid residues are organized in the plurality of synthetic peptides in random sequences.

According to some embodiments, the cationic amino acid residues are selected from the group consisting of lysine, arginine, histidine, di-amino butyric acid (Dab), ornithine, and a combination thereof. Each possibility represents a separate embodiment of the invention. According to a certain embodiment, the cationic amino acid residue is lysine.

According to additional embodiments, the hydrophobic amino acid residues are selected from the group consisting of leucine, phenylalanine, tryptophan, valine, alanine, isoleucine, glycine, tyrosine, and any combinations thereof. Each possibility represents a separate embodiment of the invention. According to some embodiments, the hydrophobic amino acid residues are selected from the group consisting of leucine, phenylalanine, tryptophan, and any combinations thereof.

According to further embodiments, the synthetic peptides comprise or consist of one species of cationic amino acid residues and one to three species of hydrophobic amino acid residues. According to yet further embodiments, the cationic amino acid residue is lysine and the hydrophobic amino acid residues are selected from the consisting of leucine, phenylalanine, tryptophan, and any combinations thereof.

According to still further embodiments, the synthetic peptides comprise or consist of one species of a cationic amino acid residues and two species of hydrophobic amino acid residues. According to yet further embodiments, the cationic amino acid residue is lysine and the hydrophobic amino acid residues are selected from the consisting of leucine, phenylalanine, tryptophan, and any combinations thereof. According to an exemplary embodiment, the hydrophobic amino acid residues are leucine and phenylalanine.

According to yet further embodiments, the synthetic peptides comprise one species of cationic amino acid residues and one species of hydrophobic amino acid residues. According to further embodiments, the synthetic peptides consist of one species of cationic amino acid residues and one species of hydrophobic amino acid residues.

According to still further embodiments, the synthetic peptides comprise lysine and leucine or lysine and phenylalanine or lysine and tryptophan. Each possibility represents a separate embodiment of the invention.

According to further embodiments, the synthetic peptides consist of lysine and leucine, lysine and phenylalanine, or lysine and tryptophan. Each possibility is a separate embodiment of the invention.

According to yet further embodiments, the ratio between the total amount of the cationic amino acid residues and the total amount of the hydrophobic amino acid residues within the plurality of synthetic peptides ranges from about 5:1 to about 1:5, alternatively the ratio ranges from about 5:1 to about 1:1, or from about 3:1 to about 1:1, or any ratio in-between. Each possibility represents a separate embodiment of the invention. According to a certain embodiment, the ratio between the total amount of the cationic amino acid residues and the total amount of the hydrophobic amino acid residues within the peptides is of about 7:3.

According to certain embodiments, the synthetic peptides comprise or consist of lysine and leucine in a ratio of about 5:1 to 1:1, alternatively in a ratio of about 3:1 to 1:1, or in a ratio of about 7:3. According to another embodiment, the mixture of the plurality of synthetic peptides comprises or consists of lysine and phenylalanine in a ratio of about 5:1 to 1:1, alternatively in a ratio of about 3:1 to 1:1, or in a ratio of about 7:3.

According to yet further embodiments, the peptides within the mixture have an identical number of amino acid residues in length. Thus, the peptides consist of 5, 6, 7, 8, 9, 10, 11, 12, 13, 14, 15, 16, 17, 18, 19, 20, 21, 22, 23, 24, 25, 30, 35, 40, or 50 amino acid residues in length or any integer in-between. According to one exemplary embodiment, the peptides consist of 10 to 30 amino acid residues in length. According to another exemplary embodiment, the peptides consist of 20 amino acid residues in length.

According to still further embodiments, the synthetic peptides comprise or consist of lysine and leucine, the ratio between the total amount of lysine and the total amount of leucine ranges from about 5:1 to about 1:1, alternatively of about 7:3, and the peptides consist of an identical number of amino acid residues ranging from 10 to 30 amino acid residues in length, alternatively of 20 amino acid residues.

According to still further embodiments, the cationic amino acid residues and the hydrophobic amino acid residues are in an L-configuration, D-configuration, or a combination thereof. According to an exemplary embodiment, the amino acid residues are all in an L-configuration.

According to additional embodiments, the linker forms a distance between the peptide and the water-insoluble polymer of about 5 Å to about 20 Å.

According to some embodiments, the peptide and the water-insoluble polymer each is independently bound to the linker via an acid stable covalent bond. According to further embodiments, the linker comprises at least two functional groups, each is independently selected from the group consisting of an amine group, a carboxylic acid group, a thiol group, a maleimide (MI) group, 6-aminohexanoic acid, an azide group, and an acetylene group. According to additional embodiments, the linker comprises at least two functional groups, each is independently selected from the group consisting of an amine group and a carboxylic acid group. According to a certain embodiment, the peptide and the water-insoluble polymer each is independently bound to the linker via an amide bond.

According to some embodiments, the linker comprises a compound selected from the group consisting of 4-hydroxymethyl-phenylacetamidomethyl (PAM), polyethylene glycol, an amino acid, and a combination thereof. According to a certain embodiment, the linker comprises PAM. According to an exemplary embodiment, the linker comprises PAM-glycine. According to another embodiment, the linker consists of 1-4 cysteine residues.

According to additional embodiments, the water-insoluble polymer is a synthetic water-insoluble polymer. According to further embodiments, the water-insoluble polymer is modified to include an amine group or a carboxylic acid group. According to yet further embodiments, the water-insoluble polymer or surface is selected from the group consisting of polystyrene, polyethylene, polycarbonate, polypropylene, polysulfone, polymethyl methacrylate, acrylonitrile, polyethylene terephthalate, polyamide, agarose, sepharose, acrylate, cellulose, cross-linked cellulose, cellulose acetate, nitrocellulose, polyvinylidene fluoride (PVDF), silica, glass, gold, and metals. Each possibility represents a separate embodiment of the invention. According to additional embodiments, the water-insoluble polymer is selected from the group consisting of polystyrene, cellulose, and cross-linked cellulose. According to a certain embodiment, the water-insoluble polymer comprises polystyrene. According to another embodiment, the water-insoluble polymer comprises polystyrene and the linker comprises PAM, such as PAM-glycine.

According to some embodiments, the mixture has tens, hundreds or up to 2ⁿ random amino acid sequences, wherein n defines the number of coupling steps in the peptide synthesis, and wherein one species of a cationic amino acid residue, such as lysine, is present in an L-configuration or D-configuration and one species of a hydrophobic amino acid residue, such as leucine, phenylalanine or tryptophan, is present in an L-configuration or D-configuration. According to further embodiments, the mixture has tens, hundreds or up to 4ⁿ random amino acid sequences, wherein n defines the number of coupling steps in the peptide synthesis, and wherein one species of a cationic amino acid residue is present in an L-configuration and D-configuration and one species of a hydrophobic amino acid residue is present in an L-configuration and D-configuration.

According to further embodiments, the water-insoluble polymer is present in a form selected from the group consisting of a bead, a sphere, a microparticle, a nanoparticle, a fiber, a mesh, a net, a web, a grid, a lattice, and any combination thereof.

Each possibility is a separate embodiment of the invention. According to an exemplary embodiment, the water-insoluble polymer is in the form of beads, such as polystyrene beads. According to another embodiment, the water-insoluble polymer is in the form of microparticles or nanoparticles.

According to another aspect, the present invention provides an antimicrobial matrix comprising a water-insoluble polymer and a mixture comprising a plurality of synthetic peptides attached thereto,
wherein the peptides consist of one species of cationic amino acid residues, one to three species of hydrophobic amino acid residues, or any combinations thereof,
wherein the ratio between the total amount of the one species of cationic amino acid residues and the total amount of the one to three species of hydrophobic amino acid residues within the mixture ranges from about 5:1 to about 1:1, and
wherein said one species of cationic amino acid residues and said one to three species of the hydrophobic amino acid residues are organized in the plurality of peptides in random sequences; according to the principles of the present invention.

According to some embodiments, the mixture consists of one species of cationic acid residues and two species of hydrophobic amino acid residues according to the principles of the present invention. According to additional embodiments, the mixture consists of one species of cationic amino acid residues and one species of hydrophobic amino acid residues according to the principles of the present invention.

According to another aspect, the present invention provides an article adapted to eliminate or exterminate viable microorganisms from a sample, the article comprising the antimicrobial matrix according to the principles of the present invention. According to some embodiments the article is selected from various solid conformations, including a column or a cartridge, or a flexible article such as a bag.

According to some embodiments, the sample is a liquid and the article is a porous or permeable bag configured to allow the liquid to enter and exit the bag, and to prevent the antimicrobial matrix to exit said bag. According to a certain embodiment, the liquid is water, preferably potable water.

According to additional embodiment, the article is a column adapted to allow a liquid, including but not limited to water, to enter the column, to contact the antimicrobial matrix, to flow through said antimicrobial matrix, and to exit said column.

According to additional embodiments, the water-insoluble polymer of the antimicrobial matrix within the article is present in a form of beads, spheres or nanoparticles. According to a certain embodiment, the water-insoluble polymer is present in the form of beads.

According to another aspect, the present invention provides a method of eliminating or exterminating viable microorganisms from a sample and/or for the purification of a sample from viable microorganisms and/or for the filtration of a sample, the method comprising a step of contacting the sample with the antimicrobial matrix according to the principles of the present invention, thereby eliminating or exterminating the viable microorganisms from said sample.

According to some embodiments, the sample is a liquid, or a semi-solid.

According to additional embodiments, the liquid as an aqueous liquid.

According to additional embodiments, the aqueous liquid is selected from the group consisting of water, fruit juice, vegetable juice, milk, dairy products, and nutritional liquids. Each possibility represents a separate embodiment of the invention.

According to further embodiments, the microorganisms are selected from the group consisting of bacteria including bacteria cysts, viruses, fungi, and yeasts. Each possibility represents a separate embodiment of the invention.

According to yet further embodiments, the bacteria are gram positive, gram negative or antibiotic-resistant bacteria.

According to some embodiment, the bacteria are selected from the group consisting of Bacillus, Enterococcus, Escherichia, Klebsiella, Listeria, Micrococcus, Mycobacteria, Staphylococcus, and Streptococcus. Each possibility represents a separate embodiment of the invention.

According to further embodiments, the bacteria are selected from the group consisting of Escherichia coli, Helicobacter, Plasmodium falciparum, Acanthamoeba, Aeromonas hydrophila, Anisakis, Ascaris lumbricoides, Bacillus cereus, Campylobacter jejuni, Clostridium botulinum, Clostridium perfringens, Cryptosporidium parvum, Cyclospora cayetanensis, Diphyllobothrium, Entamoeba histolytica, Enterococcus faecalis, Eustrongylides, Giardia lamblia, Klebsiella aerogenes, Listeria monocytogenes, Nanophyetus, Plesiomonas shigelloides, Salmonella, Shigella, Staphylococcus aureus, Streptococcus, Trichuris trichiura, Vibrio cholerae, Vibrio parahaemolyticus, Vibrio vulnificus, Yersinia enterocolitica, and Yersinia pseudotuberculosis.

According to additional embodiments, the antibiotic-resistant bacteria are multi-resistant Staphylococcus aureus (MRSA), vancomycin-resistant Enterococcus (VRE), and Pseudomonas aeruginosa. Each possibility represents a separate embodiment of the invention.

According to an exemplary embodiment, the bacteria are E. coli, Enterococcus faecalis, Klebsiella aerogenes, and Pseudomonas aeruginosa.

These and other embodiments of the present invention will be better understood in relation to the figures, description, examples and claims that follow.

### BRIEF DESCRIPTION OF THE FIGURES

FIG. 1 shows the synthesis of random sequence peptides mixture coupled to beads. Fmoc-based solid-phase synthesis methods were employed on several types of polystyrene beads using a mixture of one species of protected hydrophobic amino acid (empty circle) and one species of protected cationic amino acid (grey circle) for each coupling step to from the bioactive beads. The result of three coupling steps is illustrated. Each bead of the solid support bears many peptide chains with different sequences.

FIG. 2 shows the antimicrobial activity of the bioactive beads against E. coli. Bioactive beads (4 μmol) containing 20-mer random sequence Leucine-Lysine peptides immobilized on PAM resin were incubated with 1 ml of E. coli suspension (10⁴ CFU/ml) for 0.5 hour, 3 hours, or 6 hours at 37° C. At the end of the incubation time periods, bacterial cell count was performed by spreading the bacterial cells on LBA plates and counting their number after overnight incubation at 37° C.

FIG. 3 shows the antimicrobial activity of the bioactive beads against E. coli as a function of the peptide density on the beads. Leucine-Lysine 20-mer peptides were immobilized on PAM resin at two loading densities: 0.4 mmol/gr or 1.1 mmol/gr. Four μmol from each of the two batches of bioactive beads were incubated with 1 ml of E. coli suspension (10⁴ CFU/ml) for 0.5 hour, 3 hours, or 6 hours at 37° C. At the end of the incubation time periods, bacterial cell count was performed by spreading the bacterial cells on LBA plates and counting their number after overnight incubation at 37° C.

FIG. 4 shows the effect of the resin support on the antimicrobial activity of the bioactive beads. PAM resin or TentaGel® resin (loading 0.4 mmol/gr) were used for immobilization of 20-mer random sequence Leucine-Lysine peptides. The bioactive beads were incubated with 1 ml of E. coli suspension (10⁴ CFU/mL) for 0.5 hour, 3 hours, or 6 hours at 37° C. At the end of the incubation time periods, bacterial cell count was performed by spreading the bacterial cells on LBA plates and counting their number after overnight incubation at 37° C.

FIG. 5 shows the effect of the resin support on the antimicrobial activity of the bioactive beads. PAM resin (loading 1.1 mmol/gr) or polystyrene aminoethyl resin (PS-NH2; loading 0.94 mmol/gr) were used for immobilization of 20-mer random sequence Leucine-Lysine peptides. The bioactive beads were incubated with 1 ml of E. coli suspension (10⁴ CFU/mL) for 0.5 hour, 3 hours, or 6 hours at 37° C. At the end of the incubation time periods, bacterial cell count was performed by spreading the bacterial cells on LBA plates and counting their number after overnight incubation at 37° C.

FIG. 6 shows the effect of the hydrophobic amino acid identity on the antimicrobial activity of the bioactive beads. Random sequence peptides of Leucine-Lysine (LK), Phenylalanine-Lysine (FK) or Tryptophane-Lysine (WK), 20-mer each, were immobilized on PAM resin. The bioactive beads (10 μmol) were incubated with 1 ml of E. coli suspension (10⁴ CFU/mL) for 0.5 hour or 1 hour at 37° C. At the end of the incubation times, bacterial cell count was performed by spreading the bacterial cells on LBA plates and counting their number after overnight incubation at 37° C.

FIG. 7 shows the effect of recycling of the bioactive beads on their antimicrobial activity. Newly prepared or “recycled” bioactive beads composed of random sequence LK 20-mer or 10-mer peptides immobilized on PAM resin were incubated with 1 ml of E. coli suspension (10⁴ CFU/mL) for 0.5 hour, 3 hours, or 6 hours at 37° C. At the end of the incubation times, bacterial cell count was performed by spreading the bacterial cells on LBA plates and counting their number after overnight incubation at 37° C. Free random sequence peptides mixtures of LK 20-mer or 10-mer were used as a control at a concentration of 40 μg/ml.

FIG. 8 shows the antimicrobial activity of the bioactive beads and of the free random sequence peptides mixture in apple juice. Bioactive beads composed of random sequence LK 20-mer peptides immobilized on PAM resin (loading 1.1 mmol/gr; final concentration of 4 mole/4 mg beads) were incubated with 1 ml of E. coli suspension (10⁴ CFU/ml) for 0.5 hour, 3 hours, or 6 hours at 37° C. in commercial natural apple juice pH 3.4. At the end of the incubation time periods, bacterial cell count was performed by spreading the bacterial cells on LBA plates and counting their number after overnight incubation at 37° C. Apple juice incubated with E. coli only was used as a control. Free random sequence peptides of LK 20-mer which were added to apple juice were used as another control.

FIG. 9 shows the antimicrobial activity of the bioactive beads in apple juice. Bioactive beads composed of random sequence LK 20-mer peptides immobilized on PAM resin (final concentration of 10 mole/10 mg beads) were incubated with 1 ml of E. coli suspension (10⁴ CFU/ml) for 30 minutes at 37° C. in commercial natural apple juice pH 3.4. At the end of the incubation period, bacterial cell count was performed by spreading the bacterial cells on LBA plates and counting their number after overnight incubation at 37° C. Apple juice incubated with E. coli only was used as a control.

FIG. 10 shows the absence of hemolytic activity of bioactive beads on red blood cells (RBCs). Bioactive beads containing random sequence LK 20-mer peptides or free random sequence LK 20-mer peptides were incubated with RBCs for 85 min and thereafter the hemolytic activity of the bioactive beads or the free peptides was determined. Incubation of RBCs in the presence of Tween 20 was used as a positive control−100% hemolytic activity. The effect of PAM beads on RBCs was also evaluated.

FIGS. 11A-B show confocal microscopy images of E. coli rp eradicated on LK 20-mer bead's surface. PAM resin (×20; FIG. 11A) or random sequence LK 20-mer peptides immobilized on beads (×40; FIG. 11B) were incubated with E. coli rp for 30 minutes in PBS buffer at 37° C. Thereafter, Live/Dead staining was performed and representative confocal microscopy images are presented. Live and dead bacteria appear in green and red, respectively.

FIG. 12 is a model of a “bioactive tea bag”. The bioactive beads are entrapped in a bag made of a dense porous material which enables transport of bacteria in and out but not the bioactive beads.

FIG. 13 shows eradication of E. coli rp by LK 20-mer bioactive beads compared to a specific tailored peptide immobilized on beads. Eradication was determined by survival assays on 10⁴ CFU/ml E. coli rp in PBS containing 10 μmol/ml immobilized random sequence LK 20-mer peptides or immobilized specific tailored LK peptide at 37° C. Log eradication=Log CFU/ml_{(Treatment)}−Log CFU/ml_{(control)}.

FIG. 14 shows eradication of different bacteria by LK 20-mer bioactive beads. Incubation of 10⁴ CFU/ml of Methicillin resistant Staphylococcus aureus (MRSA), Bacillus subtilis 3610, Pseudomonas aeruginosa PAO1, or E. coli rp was carried out in PBS at 37° C. for 1 hour, with 10 μmol/mL immobilized random sequence LK 20-mer peptides.

FIGS. 15A-B show the sensitivity of E. coli K12 BW25113 mutants to LK 20-mer beads and to free random sequence peptides as compared to the wild type (WT). FIG. 15A, sensitivity of E. coli K12 BW25113 WT and mutant strains to random sequence LK 20-mer peptides immobilized on beads (10 μmol/ml). FIG. 15B, Sensitivity of E. coli K12 BW25113 WT and mutant strains to free random sequence LK 20-mer peptides (3 μg/ml) at 37° C. for 30 min incubation.

FIG. 16 shows E. coli rp eradication by a LK 20-mer bioactive beads column over time. PBS inoculated with 10⁴ CFU/ml E. coli rp was pumped in a constant flow rate of 2.5 ml/min through a column containing 5% bioactive beads in sand. Log eradication=Log CFU/ml (Treatment)−Log CFU/ml (Control).

### DETAILED DESCRIPTION OF THE INVENTION

The present invention provides an antimicrobial matrix which comprises a water-insoluble polymer and a mixture comprising a plurality of random-sequence synthetic peptides, the random-sequence synthetic peptides comprise cationic amino acid residues and/or hydrophobic amino acid residues, said peptides are linked, attached or immobilized via a linker or directly to the water-insoluble polymer.

### Antimicrobial Matrix

The present invention provides an antimicrobial matrix comprising a water-insoluble polymer, a linker, and a mixture comprising a plurality of synthetic peptides attached to the water-insoluble polymer via the linker,
wherein the peptides comprise cationic amino acid residues, hydrophobic amino acid residues, or any combinations thereof,
wherein the ratio between the total amount of the cationic amino acid residues and the total amount of the hydrophobic amino acid residues of the peptides ranges between about 10:1 to about 1:10, and
wherein the cationic amino acid residues and the hydrophobic amino acid residues are organized in the plurality of peptides in random sequences.

According to some embodiments, the plurality of synthetic peptides comprise or consist of one species of a cationic amino acid residue, one to three species of hydrophobic amino acid residues, or any combinations thereof, wherein the ratio between the total amount of the one species of cationic amino acid residue and the total amount of the one to three species of hydrophobic amino acid residue ranges from about 10:1 to about 1:10, alternatively from about 5:1 to 1:5, further alternatively from about 5:1 to 1:1, and further alternatively from about 3:1 to 1:1, and wherein the one species of said cationic amino acid residue and the one to three species of said hydrophobic amino acid residues are organized in the plurality of peptides in random sequences. According to a certain embodiment, the ratio between the total amount of one species of a cationic amino acid residue and the total amount of one to three species of hydrophobic amino acid residues within the peptides is of about 7:3.

According to additional embodiments, the plurality of synthetic peptides consist of one species of a cationic amino acid residue, one species of a hydrophobic amino acid residue, or a combination thereof, wherein the ratio between the total amount of said one species of cationic amino acid residue and the total amount of said one species of hydrophobic amino acid residue within the peptides ranges from about 10:1 to about 1:10, alternatively from 5:1 to 1:5, further alternatively from 5:1 to 1:1, and further alternatively from 3:1 to 1:1, and wherein the one species of a cationic amino acid residue and the one species of a hydrophobic amino acid residues are organized in the plurality of peptides in random sequences. According to a certain embodiment, the ratio between the total amount of one species of a cationic amino acid residue and the total amount of one species of a hydrophobic amino acid residue within the peptides is of about 7:3.

The terms “antimicrobial” or “antimicrobial activity” as used herein refers to the ability to prevent, inhibit, reduce or destroy one or more viable microorganisms. The antimicrobial activity typically refers to a lytic activity against viable microorganisms. According to some embodiments, the antimicrobial activity refers to antibacterial or bactericidal activity. However, activity against other pathogenic organisms such as viruses, fungi, yeasts, mycoplasma, and protozoa is also contemplated in the present invention.

The term “plurality” of peptides as used herein refers to at least tens, alternatively to at least hundreds, further alternatively to at least thousands of peptides. Each possibility represents a separate embodiment of the invention. According to a certain embodiment, the mixture of synthetic peptides includes millions of peptides.

The water-insoluble polymer serves as a solid support for attachment of the random-sequence peptides. The water-insoluble polymer allows performing a continuous and/or repetitive contact of samples containing the microorganisms with the random-sequence peptides attached to the polymer, as well as maintaining the random-sequence peptides affixed, thus eliminating loss of the peptides due to leaching or detachment.

The term “water-insoluble” polymer as used herein refers to a compound that typically has solubility in water of less than 1 gr/10 L at room temperature.

The phrase “water-insoluble polymer” as used herein refers to synthetic or natural polymeric materials to which coupling, linking, immobilization or attachment of the peptides through a linker is possible. According to some embodiments, the water-insoluble polymers are synthetic water-insoluble polymers. Examples of water-insoluble polymers include, but are not limited to, polystyrene, polyethylene, polycarbonate, polypropylene, polysulfone, polymethyl methacrylate, acrylonitrile, polyethylene terephthalate, polyamide (e.g., nylons); chromatography materials, such as, for example, agarose, sepharose, acrylate; filter materials or membranes such as, for example, cellulose or cross-linked cellulose, cellulose acetate, nitrocellulose, polyvinylidene fluoride (PVDF). Other water-insoluble substances which are encompassed in the present invention include silica, glass, gold, and metals, such as, for example, aluminum, chrome, titanium, and iron. Any water-insoluble polymer or substance which can be modified to include an amine group or a carboxylic acid group is useful in practicing the present invention.

Polystyrene is a polymer made from the monomer styrene, a liquid hydrocarbon that is commercially manufactured from petroleum. At room temperature, polystyrene is normally a solid thermoplastic, but can be melted at higher temperatures for molding or extrusion, and then re-solidified. Substituted styrene can be used to form an aromatic polymer with a variety of free functional groups along the polymeric chain.

The water-insoluble polymer or surface can have a form which is selected from the group consisting of a bead, a sphere, a microparticle, a nanoparticle, a fiber, a tube, a mesh, a net, a web, a grid, a lattice, a screen, a flat surface, and any combination thereof.

According to some embodiments, the water-insoluble polymer comprises a granular and/or porous substance or mixture of substances, which can allow a relatively free flowing of a solution therethrough.

The terms “attached” or “linked” as used interchangeably herein refer to any mode of contact between the water-insoluble polymer and the random-sequence peptides, which achieves immobilization of the peptides on and/or within the polymer, thus rendering the peptides insoluble or immobilized, thereby reducing or preventing peptide detachment or leakage to essentially negligible or acceptable amounts according to regulatory standards. According to some embodiments, peptide detachment is avoided. According to some embodiments, the attachment of the random-sequence peptides to the water-insoluble polymer is via a linker. According to some embodiments, the attachment of the random-sequence peptide and the water-insoluble polymer is via at least two functional groups, one of the peptide and the other of the polymer, or by any method known to immobilize peptides to a water-insoluble polymer, including but not limited to gluing, coating, such that peptide detachment is essentially negligible.

The term “linker” as used herein refers to a molecule that is interposed between the peptide and the water-insoluble polymer, so that the peptide is not directly attached to the polymer, but via the linker.

Thus, immobilization of the random-sequence peptides on and/or within the water-insoluble polymer is affected through a linker by attachment via chemical bonding interactions, including covalent bonds, metal-mediated complexation, affinity-pair bonding, and the like. According to a preferred embodiment, the binding of the peptide to the linker and the binding of the linker to the water-insoluble polymer is via covalent bonds.

Examples of covalent bonds that can be formed between the linker and the peptide, or between the water-insoluble polymer and the linker include, but are not limited to, an amide bond: R—CO—NH—R′; a thioether bond: R—S—CH₂—R′; a carbon-carbon covalent bond: C—C; and a carbon-nitrogen bond: CR₂—NH—CR′₂—. Additional possible bonds include an azide-alkyne bond, and a hydrazine-aldehyde bond.

According to an exemplary embodiment, the bond between the peptide and the linker and the bond between the linker and the water-insoluble polymer comprises an acid stable covalent bond, e.g., an amide bond.

The term “acid stable” bond as used herein refers to a bond which can withstand acid treatment, such as, for example, 50% TFA in DCM for 10 min, with limited detachment of the bound peptides, namely detachment or release of 0.1% or less of the bound peptides, alternatively of 0.05% or less of the bound peptides, further alternatively of 0.02% of the bound peptides, and yet further alternatively of 0.01% or less of the bound peptides. Each possibility represents a different embodiment of the invention.

As indicated herein, acid stable covalent bonds between the linker and the peptides and between the linker and the water-insoluble matrix are the preferred form of bonds. However, in addition to these covalent bonds, other chemical and/or physical bonds, such as hydrophobic bonds, hydrogen-hydrogen bonds, etc. can exist between the linker and the peptides and/or between the linker and the water-insoluble matrix.

Various linkers can be used to attach the peptides to the water-insoluble polymer. According to the principles of the present invention, the linker should form a distance between the peptide and the water-insoluble polymer of at least 5 Å up to 20 Å. Such a distance endows the peptides with sufficient conformational flexibility to interact with the membranes of microorganisms. Examples of linkers include, but are not limited to, hydroxymethyl-phenylacetamidomethyl (PAM), polyethylene glycol (PEG), an amino acid, poly-acrylamide, poly-methacrylic acid, a co-polymer of methacrylic acid and other acrylate monomer, poly-maleic anhydride or copolymer of (ethylene) and (maleic anhydride), and a combination thereof. According to some embodiments, the amino acid is selected from the group consisting of glycine, cysteine, serine, and a combination thereof. According to some embodiments, the linker comprises hydroxymethyl-phenylacetamidomethyl (PAM), an amino acid, or a combination thereof. According to an exemplary embodiment, the linker comprises hydroxymethyl-phenylacetamidomethyl (PAM). According to another embodiment, the linker comprises PAM covalently attached to an amino acid, such as for example, glycine.

The linker can be, for example, a bi-functional moiety, namely a compound having at least two functional groups which are capable of forming covalent bonds with functional groups of both the polymer and the peptide. Peptide attachment to the linker by covalent bonding is based on coupling of at least two functional groups, one of the linker and the other of the peptide. Linker attachment to the water-insoluble polymer by covalent bonding is also based on coupling of at least two functional groups, one on or within the polymer and the other of the linker.

Thus, the linker should have at least two functional groups, each being independently selected from the group consisting of an amine group, a carboxyl group, a thiol group, a maleimide (MI) group, 6-aminohexanoic acid, an azide group, and an acetylene group.

The water-insoluble polymer and the peptide should also independently have at least one functional group being selected from the group consisting of a carboxyl group, an amine group, a thiol group, a maleimide (MI) group, 6-aminohexanoic acid, an azide group, and an acetylene group.

Non-limiting examples of functional groups which can be utilized for coupling of a peptide with a linker include functional groups derived from the C-terminus or the N-terminus of the peptide, and functional groups derived from side chains of certain amino-acid residues. These include, for example, a carboxylic acid group of an amino acid or an amine group (stemming from the α amino group of an amino acid or from the side-chain of a cationic amino acid, e.g., lysine or arginine). According to a certain embodiment, the random-sequence peptides are linked to the linker via an amide bond through the C-terminus of the peptide or through the a carboxylic acid group of the first amino acid attached to the linker to synthesize the random sequence peptide (see the Examples below). The amide bond is non-cleavable and acid stable, i.e., stable under acidic conditions of 50% TFA in DCM for 10 min.

The linker and/or the peptides and/or the water-insoluble polymer can be modified to introduce a suitable functional group into a selected position as known to a person skilled in the art. Thus, according to an exemplary embodiment, the linker is modified by attaching to the linker an amino acid, e.g., glycine, so that the peptides being linked to the modified linker by an amide bond at their C-terminus. Alternatively, the peptides can be linked to a modified linker by an amide bond at their N-terminus or at an amine group of a side chain.

According to an exemplary embodiment, the linker has two functional groups, each is independently selected from the group consisting of an amine group and a carboxylic acid group. Thus, the linker is bound to the water-insoluble polymer via a first amide bond and to the random sequence peptide via a second amide bond, such that the peptides are immobilized to the water-insoluble polymer and are stable at acidic conditions, which conditions are typically used to detach peptides from their support at the end of the synthesis.

The linkers may also be multivalent molecules, such as polymers prepared by graft polymerization, or branched polymers having a multitude of functional groups, to which the peptides may be later attached.

The random-sequence peptides of the present invention comprise natural and non-natural cationic amino acids and hydrophobic amino acids or analogs thereof. Cationic or positively charged amino acids as used herein are selected from cationic or positively charged amino acids as known in the art. Cationic amino acids include, but are not limited to, lysine, arginine, histidine, di-amino butyric acid (Dab), and ornithine. Hydrophobic amino acids as used herein are selected from hydrophobic amino acids as known in the art. Hydrophobic amino acids include, but are not limited to, leucine, phenylalanine, tryptophan, tyrosine, isoleucine, glycine, alanine, and valine. The amino acids can be in the L-configuration, D-configuration or a combination thereof.

The term “random-sequence peptide” as used herein refers to a peptide, the amino acid sequence of which is not a tailored or predicted amino acid sequence. The amino acid sequence of a random-sequence peptide is different from the amino acid sequence of at least one peptide, alternatively of at least 2, 3, 4, 5, 10, 100, 10³, 10⁴, or more peptides, or any number in-between, in the mixture. In other words, a peptide in the mixture of the present invention is not and cannot be identical in its amino acid sequence to all the other peptides. Thus, according to some embodiments, the mixture can have up to 2ⁿ amino acid sequences, wherein n defines the number of coupling steps in the peptide synthesis, if one species of a cationic amino acid residue and one species of a hydrophobic amino acid residue are present in an L-configuration or D-configuration. According to further embodiments, the mixture can have up to 4ⁿ amino acid sequences, wherein n defines the number of coupling steps in the peptide synthesis, if one species of a cationic amino acid residue and one species of a hydrophobic amino acid residue are present in an L-configuration and D-configuration. Thus, the number of random-sequence peptides in a mixture is dictated by the length of the peptides synthesized, the various species of amino acids, and the configuration of the amino acids.

According to the invention, the random-sequence peptides comprise or consist of hydrophobic and/or cationic amino acids. According to some embodiments, one or more peptides in the mixture, preferably 10% or less, such as less than 1% of the peptides in the mixture, comprise or consist of hydrophobic amino acids but are devoid of cationic amino acids, one or more peptides in the mixture, preferably about 10% or less, such as less than 1% of the peptides in the mixture, comprise or consist of cationic amino acids but are devoid of hydrophobic amino acids, and at least about 80% of the peptides in the mixture, alternatively at least about 90%, 95%, or preferably at least about 99% of the peptides in the mixture comprise or consist of a combination of cationic and hydrophobic amino acids. According to a certain embodiment, the peptides of the mixture comprise or consist of a combination of hydrophobic and cationic amino acid residues.

The peptides of the present invention can further comprise additional amino acid residues. The peptides can comprise one or more cysteine residues at their N-terminus or C-terminus. Additionally or alternatively, the peptides can further comprise, for example, polar or uncharged amino acid residues, e.g., asparagine and/or serine, as long as the ratio between the cationic amino acid residues and the hydrophobic amino acid residues is maintained between about 10:1 to 1:10.

The term “about” as used herein refers to ±10% of the indicated numerical value.

According to some embodiments, the ratio between the cationic amino acid residues and the hydrophobic amino acid residues is about 5:1 to about 1:1, alternatively the ratio is about 3:1 to 1:1, such as about 7:3.

According to some embodiments, the present invention provides an antimicrobial matrix comprising a water-insoluble polymer, a linker, and a mixture comprising a plurality of synthetic peptides attached to the water-insoluble polymer via the linker,
wherein the peptides consist of lysine, leucine, or a combination thereof,
wherein the ratio between the total amount of lysine and the total amount of leucine ranges from about 5:1 to about 1:1, alternatively the ratio ranges from about 3:1 to 1:1, or the ratio is about 7:3,
wherein lysine and leucine are organized in the plurality of peptides in random sequences,
wherein the peptides consist of an identical number of amino acid residues, ranging from 10 to 30 amino acid residues in length, preferably 20 amino acid residues in length, and
wherein the peptide and the water-insoluble polymer each is independently bound to the linker via an amide bond.

According to a certain embodiment, the linker comprises PAM, preferably PAM-glycine. According to another embodiment, the water-insoluble polymer is polystyrene.

According to some embodiments, the process by which the peptides of the present invention are produced is performed on a water-insoluble polymer to which a linker is attached and the peptides are made by incremental elongation methods while attached to the linker, namely by a solid phase peptide synthesis method (see Examples below). Alternatively, the peptides can be synthetized on any resin or support by a solid phase peptide synthesis method, cleaved or released from the resin after synthesis, and thereafter reacted with the linker attached to the water-insoluble polymer to form the antimicrobial matrix of the present invention. Alternatively, the peptides and the linker can be synthesized on any resin or support by a solid phase peptide synthesis method, wherein the linker can be, for example 1-4 amino acid residues long of glycine or cysteine located at the N- or C-terminus of the peptides, the peptides can be cleaved or released from the resin after synthesis, and thereafter reacted with the water-insoluble polymer to form the antimicrobial matrix of the present invention.

The random-sequence peptides of the present invention are not conjugated or attached to a fatty acid. The antimicrobial activity of the antimicrobial matrix of the present invention does not require a free or conjugated fatty acid.

The antimicrobial matrix can further comprise additional agents known to have antimicrobial activity, preferably bactericidal activity, including, but not limited to, natural peptides such as host-defense peptides (HDPs), synthetic peptides, proteins or fragments thereof known to have antimicrobial activity; antibiotics; said additional agents can be attached or immobilized to the water-insoluble matrix.

The present invention further provides a process for preparing an antimicrobial matrix comprising covalently attaching a mixture of a plurality of random-sequence peptides to a water-insoluble polymer via a linker. The preparation of the antimicrobial matrix can be performed by several methods, for example, (i) by attaching a linker (L) to a water-insoluble polymer (P) to obtain a polymer-linker (P-L) moiety, and then synthesizing the mixture of random-sequence peptides (RSPs) on the polymer-linker moiety to obtain the antimicrobial matrix as described in the Examples herein below; (ii) by attaching a linker (L) to a water-insoluble polymer (P) to obtain a polymer-linker (P-L) moiety, and then attaching a pre-made mixture of random-sequence peptides to obtain the antimicrobial matrix; (iii) by synthesizing a mixture of random-sequence peptides (RSPs), which include a peptide linker, i.e., 1-4 amino acids at the N- or C-terminus of the peptides, on a resin to obtain random-sequence peptides-linker (RSPs-L) moiety, then releasing the peptides-linker moiety from the resin, and then attaching the peptides-linker (RSPs-L) moiety to the water-insoluble polymer (P) to obtain the antimicrobial matrix.

### Uses of the Antimicrobial Matrix

According to another aspect, the present invention provides a method for eliminating or exterminating one or more types of viable microorganisms from a sample, e.g., a liquid sample, said method does not involve harsh conditions which affect the characteristics of the sample. Thus, the present invention provides a method of eliminating or exterminating one or more types of viable microorganisms from a sample, the method comprises a step of exposing or contacting a sample, e.g. a liquid sample, with the antimicrobial matrix of the present invention, thereby eliminating or exterminating the one or more types of viable microorganisms.

According to additional embodiments, the present invention provides a method of eliminating or exterminating one or more types of viable microorganisms from a sample, the method comprises the following steps:
(i) exposing or contacting a sample with the antimicrobial matrix according to the principles of the present invention; and
(ii) collecting the sample.

The present invention further provides a method of eliminating or exterminating one or more types of viable microorganisms from a sample, the method comprises the following steps:
(i) exposing or contacting a first sample with the antimicrobial matrix according to the principles of the present invention;
(ii) collecting the first sample; and
(iii) washing the antimicrobial matrix.

According to some embodiments, the antimicrobial matrix can be used, for example, 2, 3, 4, 5, 6, 7, 8, 9, 10 or more times and thus steps (i) to (iii) can be repeated as many times as required as long as the antimicrobial matrix maintains the antimicrobial activity. It is to be appreciated that the cycles of contacting, collecting, and washing, i.e., steps (i) to (iii), if repeated 2-10 times or even more, improve the antimicrobial activity of the matrix.

The present invention encompasses a method of eliminating or exterminating viable microorganisms from a liquid sample when the liquid sample is contacted with the antimicrobial matrix under flow.

The term “sample” as used herein refers to all liquids or semi-solid food products from which viable microorganisms are to be eliminated or exterminated. Examples of liquids include, but are not limited to, water, fruit or vegetable juices, milk, nutritional liquids, beverages, and other aqueous solutions. Each possibility represents a separate embodiment of the invention. Examples of semi-solid food products include, but are not limited to, dairy products. According to some embodiments, a sample refers to edible solid products, such as chilled food, for example, meat, ready to cook meals, and the like.

The terms “eliminating” or “exterminating” of microorganisms as used herein means complete or partial reduction of the number of viable microorganisms from the sample material. In drinking water and food products, the heterotrophic plate count (HTC) test is typically used to determine bacteriological quality. When referring to drinking water, the Environmental Protection Agency (EPA) has set forth minimum standards for acceptance of a device proposed for use as a microbiological water purifier. Common coliforms, represented by the bacteria E. coli and Klebsiella terrigena, must show a minimum 6-log reduction, 99.9999% of organisms removed, from an influent concentration of 1×10⁷/100 ml. Common viruses, represented by poliovirus 1 (LSc) and rotavirus (Wa or SA-11) must show a minimum 4 log reduction, 99.99% of organisms removed, from an influent concentration of 1×10⁷/L. Cysts, such as those represented by Giardia muris or Giardia lamblia, are widespread, disease-inducing, and resistant to chemical disinfection. Devices that claim cyst removal must show a minimum 3 log reduction, 99.9% of cysts removed, from an influent concentration of 1×10⁶/L or 1×10⁷/L, respectively. Thus, the methods of the present invention are aimed at eliminating one or more types of viable microorganisms so as to lower the amount of the microorganisms in a sample from unacceptable levels to acceptable contamination levels according to standards determined for any given sample. The methods of the present invention encompass methods for the purification of samples, e.g., liquids, so as to eliminate or exterminate viable microorganisms, and thereby purifying said samples; methods for the filtration of samples, e.g., liquids; and/or methods of preventing microbial growth in samples, which in some embodiments include preventing biofilm formation and/or disrupting biofilms.

Techniques for detecting microorganisms are well known in the art and include, but are not limited to, plating and visual inspection to determine colony forming unit (CFU), and DNA amplification (PCR and real-time-PCR) techniques.

The term “microorganisms” is used to describe microscopic unicellular organisms which belong to bacteria, viruses, fungi, yeast, algae, and other parasites.

The term “pathogenic microorganism” is used to describe any microorganism which can cause a disease or disorder in a higher organism, such as mammals, in general, and human beings, in particular.

According to some embodiments, the pathogenic microorganisms are pathogenic bacteria, such as gram-negative bacteria, gram-positive bacteria, and antibiotic-resistant bacteria. Each possibility represents a separate embodiment of the invention.

Gram-positive bacteria are surrounded by a cell wall containing polypeptides and polysaccharides. Gram-positive bacteria include, but are not limited to, the genera Actinomyces, Bacillus, Listeria, Lactococcus, Staphylococcus, Streptococcus, Enterococcus, Mycobacterium, Corynebacterium, and Clostridium. Each possibility represents a separate embodiment of the invention.

According to some embodiments, the Staphylococcus or Streptococcus bacteria are selected from the group consisting of Staphylococcus aureus, Staphylococcus simulans, Streptococcus suis, Staphylococcus epidermidis, Streptococcus equi, Streptococcus equi zoo, Streptococcus agalactiae (GBS), Streptococcus pyogenes (GAS), Streptococcus sanguinis, Streptococcus gordonii, Streptococcus dysgalactiae, Group G Streptococcus, Group E Streptococcus, and Streptococcus pneumonia. Each possibility represents a separate embodiment of the invention.

Non-limiting examples of pathogenic microorganism are Escherichia coli, Klebsiella aerogenes, Enterococcus faecalis, Pseudomonas aeruginosa, Helicobacter, Plasmodium falciparum and related malaria-causing protozoan parasites, Acanthamoeba and other free-living amoebae, Aeromonas hydrophila, Anisakis and related worms, Ascaris lumbricoides, Bacillus cereus, Campylobacter jejuni, Clostridium botulinum, Clostridium perfringens, Corynebacterium, Cryptosporidium parvum, Cyclospora cayetanensis, Diphyllobothrium, Entamoeba histolytica, Eustrongylides, Giardia lamblia, Listeria monocytogenes, Nanophyetus, Plesiomonas shigelloides, Salmonella, Shigella, Trichuris trichiura, Vibrio cholerae, Vibrio parahaemolyticus, Vibrio vulnificus and other vibrios, Yersinia enterocolitica and Yersinia pseudotuberculosis. Each possibility represents a separate embodiment of the invention.

Pathogenic bacteria species include spore-forming Bacillus species, and spore-forming Clostridium species. Spore-forming Bacillus species cause anthrax and gastroenteritis. Spore-forming Clostridium species are responsible for botulism, tetanus, gas gangrene and pseudomembranous colitis. Corynebacterium species cause diphtheria, and Listeria species cause meningitis.

According to additional embodiments, the pathogenic bacteria include antibiotic-resistant bacteria, such as antibiotic-resistant Staphylococcus bacteria and antibiotic-resistant Streptococcus bacteria. According to further embodiments, the antibiotic-resistant Staphylococcus or Streptococcus bacteria are selected from the group consisting of methicillin-resistant Staphylococcus aureus (MRSA), vancomycin resistant Staphylococcus aureus (VRSA), daptomycin-resistant Staphylococcus aureus (DRSA), and linezolid-resistant Staphylococcus aureus (LRSA). Each possibility represents a separate embodiment of the invention.

According to some embodiments, the viruses are bacteriophage MS2, poliovirus, and rotavirus.

According to some embodiments, the sample can be water, from various sources and for various uses, wherein the sanitary condition thereof is of interest. Such water includes, but not limited to, potable water, irrigation water, reservoir water, natural source water (e.g., a spring, a well, a running stream, and a lake), swimming pool water, hot-tub water, fountain water, and the like, as well as industrial and/or household sewage, wastewater, spent water and the like. According to a certain embodiment, the aqueous solution is potable water.

Thus, according to some embodiments, the present invention provides a method for eliminating or eradicating viable microorganisms, e.g., bacteria and viruses, from a sample, e.g., a liquid such as potable water, the method comprising a step of contacting the sample with one or more antimicrobial matrices according to the principles of the present invention, thereby eliminating or eradicating the viable microorganisms from the potable water. According to further embodiments, the antimicrobial matrix comprises a mixture of a plurality of synthetic peptides consisting of lysine and leucine attached to a water-insoluble polymer through a linker, wherein the ratio between the total amount of lysine and the total amount of leucine within the peptides ranges from about 3:1 to 1:1, alternatively the ratio is about 7:3, wherein the peptides are of 20 amino acid residues in length, and wherein the peptide and the water-insoluble polymer each is independently bound to the linker via an amide bond. According to some embodiments, the water-insoluble polymer is in the form of beads, nanoparticles, or spheres. According to an exemplary embodiment, the linker comprises PAM, e.g., PAM-glycine.

It is to be understood that the antimicrobial matrix can include one or more of the antimicrobial matrices of the present invention. For example, a first antimicrobial matrix comprising a mixture of random sequence peptides consisting of leucine and lysine can be used in combination with a second antimicrobial matrix comprising a mixture of random sequence peptides consisting of phenylalanine and lysine and/or with a third antimicrobial matrix comprising a mixture of random sequence peptides consisting of tryptophan and lysine, etc.

The random-sequence peptides can be attached on and/or in the physical structural elements of the water-insoluble polymer. In cases where the structural elements of the polymer are granular but not porous, such as, for example, in cases where the water-insoluble polymer is made of solid spheres, beads or particles, the random-sequence peptides are attached on the surface of the beads or particles via a linker, and a liquid that flows between the beads or particles comes in contact with the peptides, thus allowing the peptides to interact with the microorganisms and to eliminate them from the liquid, probably by disrupting their membranes and lysis, as described in the Examples below.

According to some embodiments, for eliminating microorganisms from drinking water, a filtration system comprising activated carbon may be used prior to or subsequent to the contact/exposure of the water with the antimicrobial matrix of the present invention. Carbon filters are generally used to improve the taste of water by removing organic materials, such as odor and taste producing compounds, synthetic industrial/agricultural compounds, from the water. The carbon filter may comprise particles or granules of carbon selected by size so as to block passage of particles exceeding a certain dimension. If water is filtered, the taste and odor producing compounds of natural and industrial origin are readily adsorbed by activated carbon resulting in taste and odor free clear drinking water. Some inorganic materials having adverse health effects including mercury, arsenic, lead and fluoride are also removed by adsorption to activated carbon. Viruses in water are also adsorbed to the activated carbon to a great degree depending upon the pH of the water.

According to another aspect, the present invention provides an article adapted to eliminate or exterminate one or more types of viable microorganisms from a sample, wherein the article comprises the antimicrobial matrix according to the principles of the present invention.

According to some embodiments, the sample is liquid. According to a certain embodiment, the liquid is water, e.g., drinking water.

According to some embodiments, the article is configured to allow a liquid, e.g., water, to enter the article, to contact the antimicrobial matrix, to flow through said antimicrobial matrix and to exit said article. Thus, according to some embodiments, the article is configured as a column with a prefiltered liquid flowing through an inlet, contacting the antimicrobial matrix in the column, and a filtered liquid flowing out of the column through an outlet. The antimicrobial matrix being enclosed within the column.

According to additional embodiments, the article is configured as a porous or permeable bag, such as a tea bag, adapted to allow liquid to enter the bag, to contact the antimicrobial matrix, and to exit the bag. The porous or permeable bag is adapted to prevent the antimicrobial matrix to exit the bag. The terms “porous bag” or “permeable bag” as used herein refer to a bag having pores of predefined size which allow movement of a liquid, bacteria and fragments thereof, and other molecules from outside the bag inside and vice versa, but does not allow such movement of the antimicrobial matrix.

According to additional embodiments, the article can be configured as a cartridge. The term “cartridge” means a component which can be actuated by a larger unit through a suitable interface. The unit can comprise one or more elements for performing other processes upstream or downstream of the cartridge. The cartridge can comprise a chamber to enclose the antimicrobial matrix.

The antimicrobial matrix of the article of the present invention being capable of capturing the viable microorganisms present in the liquid, thereby providing purified or filtered liquid, which contains acceptable levels of viable microorganisms.

According to some embodiments, the antimicrobial matrix of the article can include one or more antimicrobial matrices.

According to additional embodiments, the water-insoluble polymer of the antimicrobial matrix is present in a form of beads, spheres, micro- or nanoparticles, a mesh, a net, a web, a grid, and/or a lattice. Each possibility represents a separate embodiment of the invention. According to further embodiments, the water-insoluble matrix is present in the form of beads, spheres or nanoparticles. According to a certain embodiment, the water-insoluble polymer is present in the form of beads. Thus, the antimicrobial matrix within a column is useful for eliminating viable microorganisms from a liquid when the liquid flows therethrough; the matrix is re-usable and can be recycled.

According to some embodiments, the sample to be contacted with the antimicrobial matrix of the present invention can be a solid food product and the antimicrobial matrix can be integrated in the food packaging material or can be immobilized to the food packaging facing the food. According to some embodiments, the solid food product can be in the form of a powder.

According to some embodiments, the sample is a health care product, and the antimicrobial matrix can be coated on the health care product packings facing the health care product.

According to some embodiments, the present invention provides a hemodialysis apparatus comprising the antimicrobial matrix of the invention, wherein the hemodialysis apparatus is configured to receive blood of a patient, the blood is contacted with said antimicrobial matrix to eliminate microorganisms present in the blood, and the treated blood is then returned to the patient.

It is to be noted that each possibility disclosed throughout the specification represents a separate embodiment of the invention.

The following examples are to be considered merely as illustrative and non-limiting in nature. It will be apparent to one skilled in the art to which the present invention pertains that many modifications, permutations, and variations may be made without departing from the scope of the invention.

### EXAMPLES

### Methods

### Synthesis of Soluble Random Sequence Peptide Mixtures

Random sequence peptide mixtures were synthesized using microwave irradiation on Rink Amide resin (Substitution 0.53 mmol/gr, 25 μmol) in Silicol filter tubes. Briefly, coupling reactions were conducted with binary combinations of L/D-protected α-amino acids, with a freshly prepared stock solution that contained the protected amino acids in 1:1 molar ratio and stereochemistry, which was used for each coupling step. Before each coupling step, an aliquot containing 4 equiv. (100 μmol) of the amino acid mixture was activated with 4 equiv. of HBTU and 8 equiv. of DIEA, in dimethylformamide (DMF). After the activated amino acid solution was added to the resin, the reaction mixture was heated to 70° C. in a MARS VI (CEM, USA) multimode microwave (2 minutes ramp to 70° C., 4 minutes hold 70° C.) with stirring. Fmoc deprotection reactions used 20% piperidine in DMF. Reaction solutions were heated to 80° C. in the microwave (2 minutes ramp to 80° C., 2 minutes hold 80° C.) with stirring. After each coupling/deprotection cycle the resin was washed 3 times with DMF. Upon completion of the synthesis, the peptide mixture was cleaved from the resin by stirring the resin in a solution containing 95% trifluoroacetic acid (TFA), 2.5% deionized water, and 2.5% triisopropylsilane for 3 hours. The peptide mixture was precipitated from the TFA solution by the addition of cold ether. The precipitated peptide mixture was collected by centrifugation. Ether was removed, and the pellet was dried and dissolved in deionized water, frozen in liquid nitrogen and lyophilized.

### Preparation of Immobilized Random Sequence Peptide Mixtures

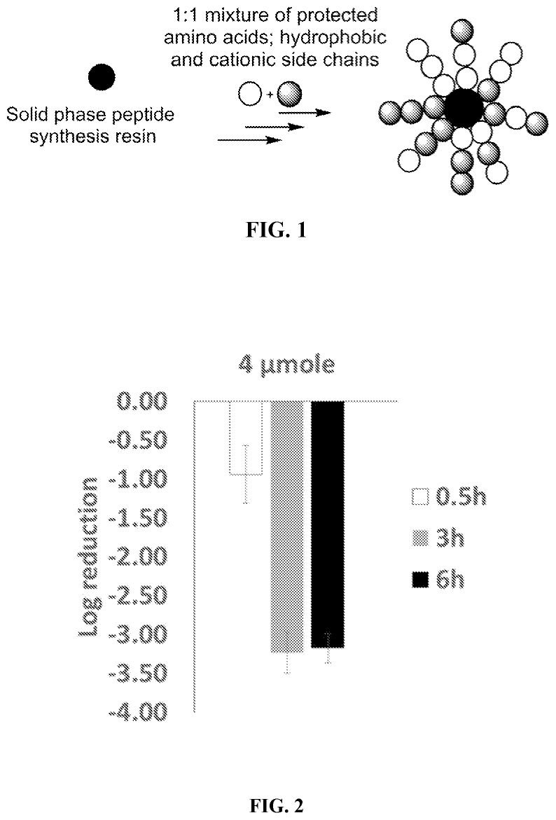

The preparation of bioactive beads, namely peptides covalently attached to a resin, was performed similarly to the synthesis of the soluble peptides mixture described herein above with one modification: the resin for synthesis of the peptides was N-Boc-glycine phenylacetamidomethyl-resin (PAM resin) or Polystyrene aminomethyl resin (PS-NH₂ resin), wherein the linkage of the peptides to the resin was stable to acid, enabling side protection group cleavage with negligible or essentially no cleavage of the peptides from the resin. Prior to peptide synthesis, Boc protecting groups of the PAM resin were removed by shaking in a 1:1 trifluoroacetic acid (TFA): dichloromethane (DCM) solution for 0.5 hour. The resin was washed with dimethylformamide (DMF) and DCM, and peptide synthesis was performed as described herein above. Upon completion of the synthesis, Boc side chain protecting groups were removed by shaking in a solution of 50% TFA, 2.5% triisopropylsilane, and 47.5% DCM for 10 minutes. Thereafter, the beads were washed with DCM and filtered to remove TFA residues. FIG. 1 is a scheme of the bioactive beads. In this method, the amount of peptides which were cleaved and released to the TFA solutions was low. The acid was evaporated under nitrogen stream and the molecular weight of the peptides was determined by MALDI TOF MS and amino acid analysis.

Alternatively, synthesis of random sequence peptides was performed on acid labile linker resins such as Rink amide resin or TentaGel® R RAM Resin with Fmoc-Leucine and Fmoc-Lysine (Mtt). The Mtt protecting group was removed by incubation in a low acid concentration solution of 1% TFA in DCM (v/v) for 10 minutes for two cycles. Under these conditions, the linker was stable and the peptide stayed immobilized on the resin.

When the polyethylene glycol (PEG) was used as a linker, prior to coupling to polystyrene amine resin, 3 equivalents PEG were activated in DMF using 4 equiv. of HBTU and 8 equiv. DIEA. Coupling of PEG to the resin was conducted over 72 hours at room temperature with agitation (150 rpm). Then, LK 20-mer synthesis, followed by Boc release, was conducted as described above.

### Bacterial Strains

For the mutants experiment, WT strain of E. coli K12 BW25113, and single-gene knockout mutants of this strain (obtained from the E. coli Keio collection and kindly received from Shimshon Belkin's lab) were used (Table 1). For survival assays, confocal microscopy analysis and antimicrobial column assay E. coli K12 MG1655 (rp) was used. Survival assays were also carried out on Methicillin-resistant Staphylococcus aureus (MRSA) and Pseudomonas aeruginosa PAO1. The WT strains were grown in lysogeny broth (LB) or LB agar plates at 37° C. The mutants were grown at the same conditions, with the addition of 50 μg/ml kanamycin. Bacteria were stored at −80° C. in 25% glycerol and at 4° C. on LB agar plate.

### Antimicrobial Activity

E. coli rp was grown in LB (Luria or lysogeny broth) under 180 rpm at 37° C. overnight. The bacteria were washed three times with phosphate buffered saline (PBS) pH 7.4 by centrifugation for removing the growth medium. Suspensions of approximately 10⁴ CFU/ml E. coli in PBS or in commercial natural pasteurized apple juice (pH 3.4) were prepared and used for the experiments. The bioactive beads were suspended in DMF and washed three times with DCM before each experiment. One ml of bacteria suspension was added to each tube and incubated at 37° C., 180 rpm in PBS with 10 μmol/ml of LK 20-mer beads. Bacteria suspensions without beads and bacteria suspensions in the presence of 40 μg/ml free random peptide mixtures were used as controls. Bacterial cell counts were performed by spreading the bacterial cells on LB agar plates and counting their number after incubation at 37° C. overnight. To examine the bactericidal activity of the beads on different bacterial strains, survival assays have been carried out on E. coli rp, Methicillin-resistant Staphylococcus aureus (MRSA) and Pseudomonas aeruginosa PAO1. E. coli rp, Methicillin-resistant Staphylococcus aureus (MRSA) and Pseudomonas aeruginosa PAO1 cultures were grown overnight in LB, and were then centrifuged and washed in PBS (pH 7.4) for three times. Suspension of about 10⁴ CFU/ml were obtained. Incubation of 1 ml bacteria suspension was performed in round bottom tubes in agitation (200 RPM) with bioactive beads in PBS buffer at 37° C. For the peptide properties comparison assays, incubation was performed with 10 μmol/ml of beads for 30 min and 1 hour. To assess eradication of MRSA and P. aeruginosa the beads' concentration was 10 μmol/ml, and samples were taken after 1 hour of incubation. Tubes containing only bacteria were used as control. After incubation, the beads submerged, samples were taken from the supernatant and the concentration of bacteria remained in the suspension was numerated by colony forming units (CFU) method on LB agar.

To examine the bactericidal activity of bioactive beads on E. coli, Klebsiella aerogenes, Enterococcus faecalis, and Pseudomonas aeruginosa, the bacteria cultures were grown overnight in LB, and were then centrifuged and washed in PBS (pH 7.4) for three times. Suspension of about 10⁵ CFU/ml were obtained. Incubation of 1 ml bacteria suspension was performed in round bottom tubes in PBS buffer at 37° C. for 20 min in agitation (200 RPM) with 10 μmol/ml 20-mer peptides immobilized on beads. The immobilized 20-mer peptides evaluated were as follows: leucine and lysine (LK) at two different ratios: 1:1 and 3:7; phenylalanine and lysine (FK) at a ratio of 1:1; and tryptophan and lysine at a ratio of 1:1. Tubes containing only bacteria were used as control. After the 20 min incubation, the beads submerged, samples were taken from the supernatant and the concentration of bacteria remained in the suspension was numerated by the colony forming units (CFU) method on selective agar plates.

Antimicrobial Activity of the Beads Against E. coli Mutants

Survival assays were carried out as described above, with some modifications. E. coli K12 BW25113 WT strain culture was grown overnight in LB, and the mutant strains were grown overnight in LB with 50 μg/ml kanamycin. The bacteria were then centrifuged and washed in PBS buffer for three times. The suspensions were diluted to obtain final bacterial concentration of ˜ 10⁶ CFU/ml. Incubation was carried out as described above, in the presence of 10 μmol/ml LK 20-mer beads. Samples were taken after 30 min of incubation and the microbial load of the suspension was numerated by CFU method on LB agar. Samples from the mutants' tubes were spread after serial dilutions in PBS on LB agar plates with 50 μg/ml kanamycin to generate CFU/mL values.

### Live/Dead Staining and Confocal Microscopy Analysis

E. coli rp culture was grown overnight in LB, and was then centrifuged and washed in PBS buffer for three times. The suspension was diluted to obtain ˜106 CFU/ml. To watch the beads after incubation with bacteria, incubation of 1 ml bacteria suspension was performed in agitation (200 RPM) with 4 μmol/ml LK 20-mer beads in PBS buffer at 37° C. for 30 min. Incubation of the bacteria with PAM resin without peptides was used as control. To explore the viability of bacteria in the supernatant over time, incubation was performed for 30 min, 1 and 3 hours, and bacteria only was used as control. After incubation, the supernatant was transferred and the remaining beads were stained using Live/Dead BacLight Bacterial Viability Kit (ThermoFisher). Each sample was stained with 2.5 μl propidium iodide (red) and 2.5 μl SYTO9 (green) and incubated for 15 minutes in the dark. Then fluorescent images were taken by 63* water lens (PMT emission 490-530 for SYTO9 and 600-644 for propidium iodide) using a Leica SP8 Confocal Laser Scanning Microscope.

### Beads Hemolysis Assay

One ml of human blood was suspended in 50 ml of 20 mM Tris buffer containing 150 mM NaCl, pH 7.2. The suspension was centrifuged (3500 RPM for 8 minutes) and washed by the buffer for three times to extract the red blood cells (RBCs). LK 20-mer beads were suspended in 0.5 ml buffer and then 0.5 ml RBC suspension was added to yield a final concentration of the beads of 10 μmol/ml. The treatments were as follows: 1) 50 μg/ml free LK 20-mer peptide; 2) 10 μmol/ml bioactive beads; 3) PAM resin not loaded with peptides; 4) Treatment with 1% Tween 20 as a positive control. The incubation continued for 85 minutes in round bottom tubes under agitation (200 RPM) at 37° C. At the end of the incubation the tubes were centrifuged (3500 RPM for 8 minutes), and 100 μl of the supernatant was transferred into 96-well plate (ThermoFisher). The hemolytic effect of the treatments was determined by measuring the absorption of the samples in 405 nm wavelength in a Tecan Infinite Pro Plate reader. The hemolysis percentage was normalized with respect to the Tween 20 control, which was determined as 100% hemolysis.

### Antimicrobial Beads Column

E. coli rp culture was grown overnight in LB, and was then centrifuged and washed in PBS buffer for three times. The suspension was diluted to obtain 1 L PBS inoculated with ˜ 10⁴ CFU/ml. A mixture of 5% LK 20-mer beads in sterile sand (250 mg beads with 4.750 g sand) was included in an antimicrobial column, 10 mm diameter and 40 mm length of active layer. Two sterile geotextile filters were placed in either end of the bead-sand mixture. The column was saturated with sterile DDW in a constant flow rate of 2.5 ml/min upwards to move captured air. The inoculated PBS was transported from downwards at a constant flow rate of 2.5 ml/min. The samples were taken after 30 min and then every hour for 10 hours. The concentration of viable bacteria in the emerging water after filtration was determined by CFU method on LB agar.

### Antiviral Activity of the Bioactive Beads

To examine the activity of the bioactive beads on bacteriophage MS2, survival assays of E. coli have been carried out. E. coli cultures were grown overnight in LB, and were then centrifuged and washed in PBS (pH 7.4) for three times. Suspension of about 10³ PFU/ml were obtained. Incubation of 1 ml bacteria suspension was performed in round bottom tubes in PBS buffer at 37° C. in agitation (200 RPM) with 10 μmol/ml 20-mer peptides immobilized on beads. The immobilized 20-mer peptides evaluated were as follows: leucine and lysine (LK) at two different ratios: 1:1 and 3:7; phenylalanine and lysine (FK) at a ratio of 1:1; and tryptophan and lysine at a ratio of 1:1. After 20 min incubation, the beads submerged, samples were taken from the supernatant, and the concentration of bacteriophage MS2 which remained in the suspension was numerated by the plaque forming units (PFU) method on agar plates.

### Example 1

### Antimicrobial Activity of Bioactive Beads

The effect of the immobilized peptides on E. coli rp was determined. E. coli cells (10⁴ CFU/ml) where incubated with 4 μmol of bioactive beads, i.e., Leucine-Lysine 20-mer peptides synthesized (immobilized) on phenylacetamidomethyl (PAM) resin. The incubation continued for 0.5 hour, 3 hours or 6 hours. FIG. 2 shows that incubation of E. coli cells with the bioactive beads caused a three-log reduction in the bacterial cell number after 3 hours of incubation.

When two different amounts of the 20-mer random sequence peptides, namely 0.4 mmol/gr or 1.1 mmol/gr, were loaded on the beads for the preparation of the bioactive beads, and the antimicrobial activity of these bioactive beads (4 μmol) on E. coli (10⁴ CFU/ml) was evaluated, the results indicated that the density of the immobilized peptides onto the surface of the beads did not affect significantly the antimicrobial activity of the bioactive beads (FIG. 3).

### Example 2

### The Effect of Various Resin Linkers on the Antimicrobial Activity of the Bioactive Beads

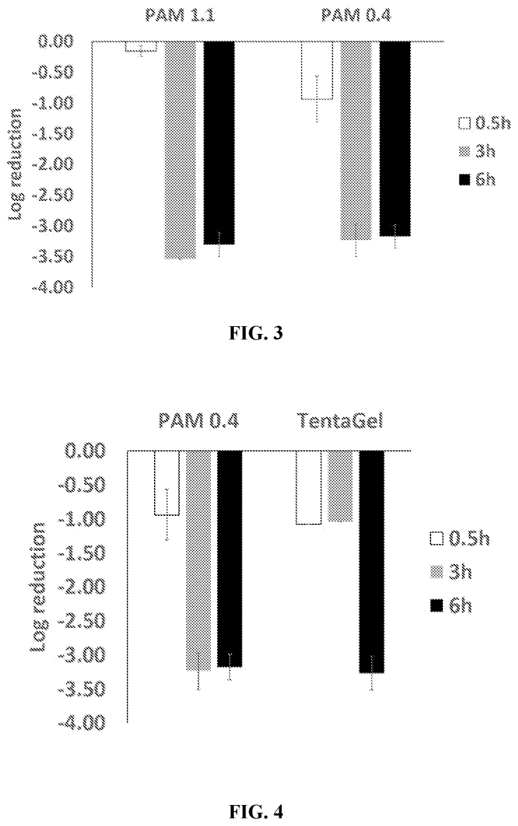

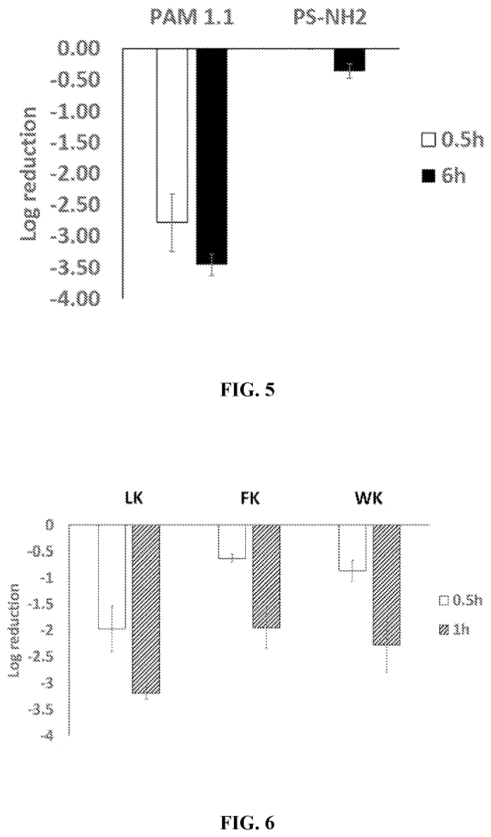

Leucine-Lysine random peptides were synthesized on polystyrene resins having different linkers and the antimicrobial activity of the peptides immobilized on the beads against E. coli was determined. As shown in FIG. 4, the peptides immobilized on phenylacetamidomethyl (PAM) resin were highly active than the peptides immobilized on TentaGel® resin, i.e., polystyrene with PEG, in reducing the number of bacterial cells after 3 hours of incubation. Also, the peptides immobilized on PAM resin were highly active than peptides immobilized on polystyrene aminomethyl resin (PS-NH₂ resin) in reducing the number of bacterial cells after 0.5 hour of incubation (FIG. 5). These results may indicate that the PAM linker plays some role in the antimicrobial activity of the bioactive beads.

### Example 3

### The Effect of Amino Acids Composition on the Antimicrobial Activity of the Bioactive Beads

The effect of two other hydrophobic amino acids on the antimicrobial activity of the bioactive beads was determined. Radom sequence peptides were synthesized with either one of the two hydrophobic amino acids: Phenylalanine (F) or Tryptophan (W), to form a binary combination with Lysine (K), and the effect of the immobilized peptides on E. coli cell number was evaluated. As seen in FIG. 6, the immobilized FK peptides or the immobilized WK peptides caused about half a log reduction in bacterial cell number after 30 min of incubation, while the immobilized LK peptides caused a two-log reduction in E. coli number after the same period of incubation.

### Example 4

### The Effect of Peptide Length and Recycling on the Antimicrobial Activity of the Bioactive Beads

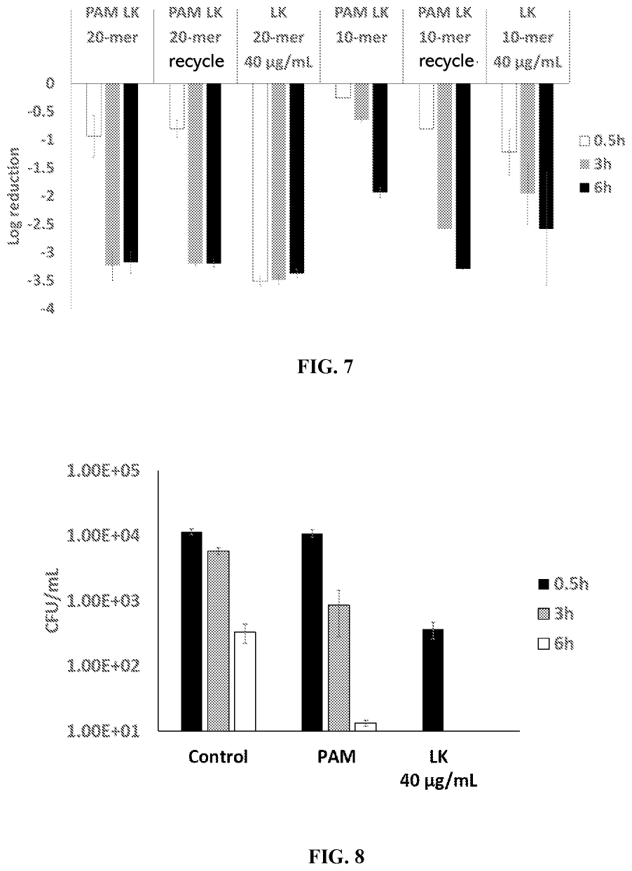

The effect of peptide length on the antimicrobial activity of the bioactive beads was next determined. LK 10-mer random sequence peptides were synthesized on PAM resin and compared to LK 20-mer random sequence peptides synthesized on PAM resin. As shown in FIG. 7, the antimicrobial activity of the immobilized LK 20-mer peptides was higher than that of the immobilized LK 10-mer peptides.

The effect of recycling of the beads on their antimicrobial activity was also evaluated. Freshly prepared bioactive beads composed of LK 20-mer peptides immobilized on PAM resin or LK 10-mer peptides immobilized on PAM resin were incubated with E. coli (10⁴ CFU/ml) for 0.5 hour, 3 hours or 6 hours. In addition, recycled bioactive beads, i.e., bioactive beads composed of LK 20-mer peptides immobilized on PAM resin or LK 10-mer peptides immobilized on PAM resin which were incubated with E. coli (10⁴ CFU/ml) once, then washed with DCM and water, and thereafter were incubated again with E. coli (10⁴ CFU/ml) for the same periods of times. At the end of the incubation times, the number of bacterial cells was determined. As seen in FIG. 7, the recycled beads were active, and interestingly, the antimicrobial activity of the recycled beads was higher than that of the freshly prepared beads both for the 10-mer and 20-mer peptides. These results indicate the re-usability of the bioactive beads. Moreover, bioactive beads that were recycled up to 10 times were shown to reduce E. coli cell number similarly as bioactive beads recycled once. Without being bound to any mechanism of action, it is suggested that the enhanced activity of the beads on second use can be associated with their exposure to an aqueous medium which enlarges their surface and thus enables more contact with bacteria.

### Example 5

### The Antimicrobial Activity of Bioactive Beads in Apple Juice

The antimicrobial activity of the bioactive beads (LK 20-mer immobilized on PAM resin; 4 μmol/ml) in apple juice was next evaluated. E. coli (10⁴ cells/ml) were inoculated with commercially available apple juice for 0.5 hour, 3 hours or 6 hours and the number of bacterial cells was determined. After 3 hours of incubation, a significant reduction in the number of bacterial cells was demonstrated (FIG. 8). The antimicrobial activity of the bioactive beads was even more pronounced after 6 hours of incubation. These results demonstrate the possible use of the bioactive beads in eliminating bacterial cells in potable or nutritional liquids.

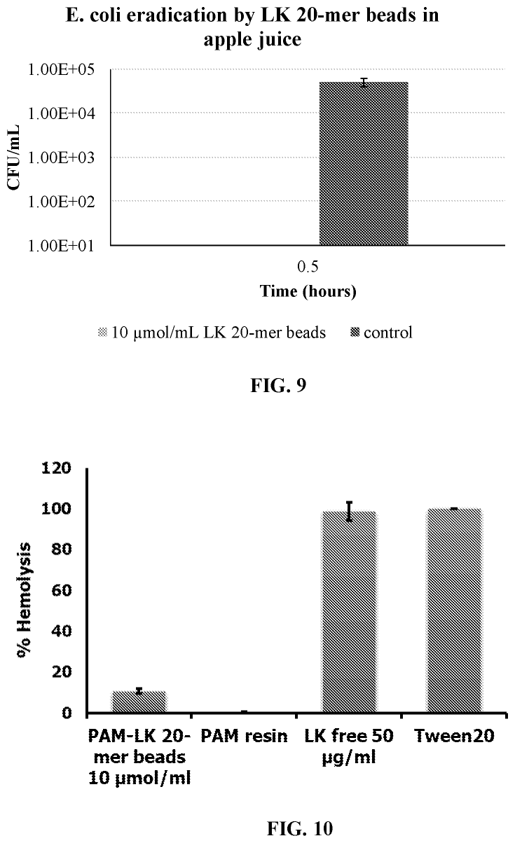

In an additional experiment, the antimicrobial activity of the bioactive beads against E. coli in apple juice was evaluated, however this time the concentration of the bioactive beads was higher (10 μmol/ml). As shown in FIG. 9, incubation of the bioactive beads with E. coli for 30 minutes resulted in a complete eradication of the bacteria.

### Example 6

### The Hemolytic Activity of Bioactive Beads

The hemolytic activity of the bioactive beads was then evaluated.

As shown in FIG. 10, while the 20-mer LK random sequence peptides were found to be highly hemolytic at relatively low concentration (50 μg/ml), immobilization of the peptides onto PAM resin reduced or even eliminated their hemolytic activity, despite the fact that the concentration of the immobilized peptides was much higher than that of the free 20-mer LK peptides (˜3 orders of magnitude higher). These results suggest that peptide self-assembly is intrinsic to the hemolytic mechanism of action.

On the other hand, the strong antibacterial or bactericidal effect and reduced hemolytic activity obtained by the immobilized peptides indicate high selectivity for bacterial cells compared to human red blood cells (RBCs), which suggests that the reduced conformational flexibility of the immobilized peptides may prevent their self-assembly and interaction with RBCs. The difference between free and immobilized random sequence peptides in selectivity to bacterial cells is an additional indication to their different mode of action.

These results imply that bioactive beads containing 20-mer LK peptides can be useful for eliminating bacteria from blood during dialysis with no harmful effect to RBCs.

### Example 7

### Mechanism of Action of Antimicrobial Beads

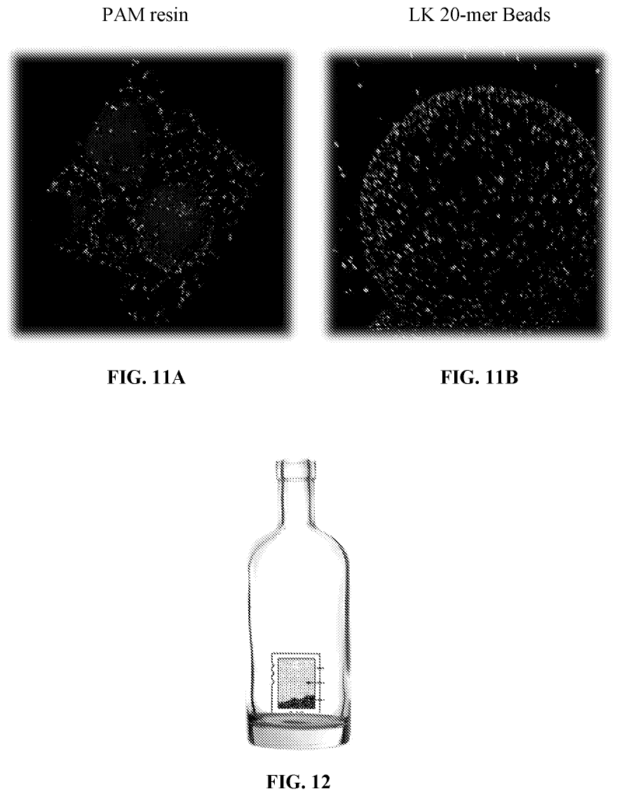

In order to evaluate the viability and distribution of E. coli cells after exposure to bioactive beads, the beads were visualized using Live/Dead staining, which contains SYTO9 (for live cells, green) and propidium iodide (for dead or injured cells, red). Representative confocal microscopy images are shown in FIGS. 11A-B. As shown in FIG. 11B, most of the dead cells (red) were localized on the surface of the beads, indicating that not only bacterial adsorption occurs, but also eradication. Some live bacteria (green) were observed on the bead, indicating that live bacteria adsorbed to the beads. Several live cells were also observed in the surrounding solution. Exposure of E. coli to PAM resin not loaded with peptides resulted in vast majority of live bacteria. The confocal microscopy images indicate that the bioactive beads display typical features of antimicrobial mechanism of action which involves bacterial adsorption and membrane disruption to achieve bacterial killing. As the random sequence peptides mixtures of the present invention are composed of cationic and hydrophobic amino acids, these peptides exhibit electrostatic and hydrophobic interactions with the hydrophobic-anionic bacterial membrane, thus adsorbing bacteria to the beads, followed by membrane disruption, bacterial cell death and finally release of the dead cells.

### Example 8

### Bioactive “Tea Bag”

The bioactive beads are trapped in a “tea bag” and inserted to a potable liquid package so as to obtain a “bioactive tea bag” as shown in FIG. 12.

### Example 9

### Antibacterial Activity of Immobilized Random Sequence Peptides Vs. Immobilized Specific Peptide

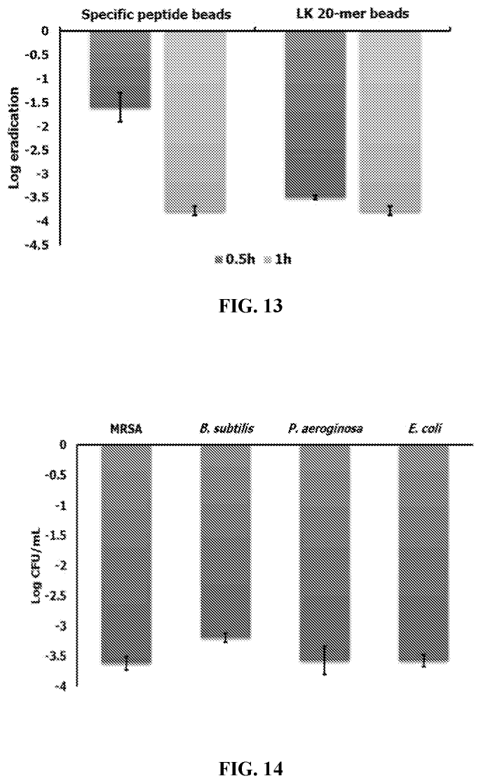

The antibacterial effect of LK 20-mer random-sequence peptides immobilized on beads was compared to that of an immobilized known peptide, a 14-mer peptide which possesses potent antimicrobial activity against the pathogenic E. coli O157:H7 strain (designated 14LKK in Haynie et al., Antimicrob Agents Chemother, 1995, 39, 301-307). As shown in FIG. 13, E. coli load was reduced by ˜3.5 orders of magnitude after 30 minutes incubation by the LK 20-mer beads, but only a ˜1.6 orders of magnitude reduction was observed for the homogeneous tailored specific peptide. These results indicate that the random sequence LK 20-mer peptides mixture immobilized on PAM resin exert faster eradication kinetics against E. coli as compared to a LK peptide of a specific amino acid sequence immobilized on the same resin.

### Example 10

### Antibacterial Activity of Bioactive Beads Against Various Bacterial Strains

To assess the ability of the LK 20-mer beads to combat a variety of bacteria, the antibacterial/bactericidal effect of the bioactive beads against both gram-positive and gram-negative bacteria was determined (FIG. 14). For that aim, eradication of the clinically relevant pathogens: methicillin-resistant Staphylococcus aureus (MRSA) and P. aeruginosa PAO1, which developed a broad multi-drug resistance, as well as of the model gram-negative bacteria E. coli rp and gram-positive bacteria B. subtilis 3610 was tested.

As shown in FIG. 14, a significant reduction in bacterial load of ˜3.6, ˜3.5, ˜3.5 and ˜3.1 orders of magnitude was achieved after one hour of incubation with the beads and MRSA, P. aeruginosa, E. coli rp, and B. subtilis, respectively. These results indicate that immobilized random-sequence peptide mixtures are active very efficiently against both gram-positive and gram-negative bacteria. This broad activity of the beads and ability to combat efficiently multi-drug resistant bacteria demonstrate their use as bactericidal agents.

The effect of LK 20-mer beads on eradiation of Alicyclobacillus spp. was next examined. Alicyclobacillus spp. are gram-positive thermophilic-acidophilic bacteria which belong to the thermo-acidophilic bacilli (TAB) group, are aerobic bacteria and are found in soil, water, fruit surfaces, and various fruit and acidic products. These bacteria are spore-forming bacteria and therefore can survive heat treatments of beverages. In addition, these bacteria can grow at a very low pH and therefore can grow in acidic beverages such as apple juice, orange juice, and sparkling beverages. Although this group of bacteria is non-pathogenic, it can spoil large amounts of food and drink products. The most commonly species associated with spoilage of acidic beverages and fruit juices is A. acidoterrestris (AAT). ATT grow at temperatures between 25° C. and 60° C. and at a pH between 2 and 6. As a result of the high survival of the spores of AAT during pasteurization, new strategies for eradicating ATT contaminations are required.

For that end, incubation of ATT with 10 μmol/mL LK 20-mer beads at 45° C. in YSG medium (containing a yeast extract, soluble starch and D-glucose) resulted not only in growth inhibition by the beads, but also in eradication of the bacterial cells. A reduction of ˜2 orders of magnitude was observed every 3 hours, while without the beads the bacteria grew to a very high bacterial load. These results indicate that the bioactive beads exhibit an efficient antibacterial activity against AAT and that these beads can be used as an effective means to eradicate or sterilize liquids from AAT and other thermal resistant bacteria, thereby avoid thermal pasteurization.

### Example 11

### Antibacterial Activity of Bioactive Beads Against Different E. coli Strains

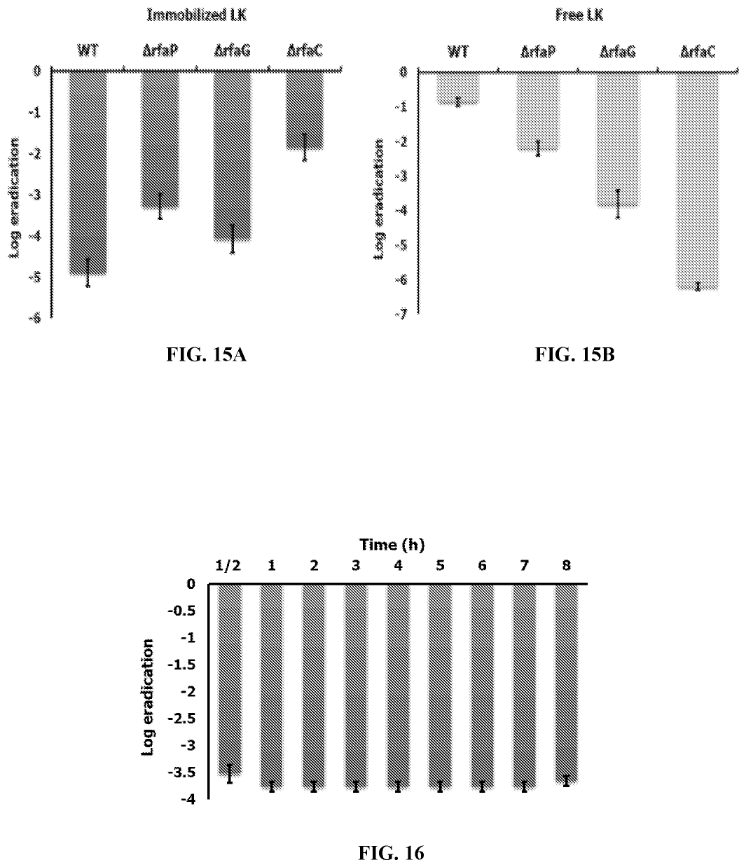

Lipopolysaccharides (LPS) on the outer membrane of Gram-negative bacteria contribute to their hydrophobicity and negative charge. The antibacterial activity of LK 20-mer beads on mutant E. coli strains with modified LPS was next evaluated (Table 1 and FIG. 15).

TABLE 1
E. coli WT and mutant strains: the function of deleted genes and the LPS
structure of each bacterium.

| | Gene | | |
|---|---|---|---|
| Strain | deletion | Function of deleted gene | Character of LPS core |
| E. coli K12 | WT | — | Intact core |
| BW25113 | | | |
| E. coli K12 | rfaP | Phosphate groups addition | Lacking the third |
| BW25113 | | to the first heptose of the | heptose and phosphate |
| | | inner core of LPS | groups of the inner |
| | | | core |
| E. coli K12 | rfaG | Involved in the addition of | Lacking outer core |
| BW25113 | | the first glucose of the LPS | |
| | | outer core | |
| E. coli K12 | rfaC | Addition of the first | Lacking outer core |
| BW25113 | | heptose of the LPS inner | and the heptoses of |
| | | core | the inner core |

As shown in FIG. 15A, all mutants exhibited lower sensitivity compared to the wild type (WT) strain where the inactivation followed the order: WT>ΔrfaG>ΔrfaP>ΔrfaC. The ΔrfaC mutant, which possesses the shortest LPS form, was the most resistant to the beads. The strains with greater negative charge (WT and ΔrfaG) were found to be more sensitive to the cationic LK 20-mer beads compared to the strains lacking two phosphate groups (ΔrfaP and ΔrfaC). This suggests an electrostatic interaction contribution. When comparing the WT to ΔrfaG strains and ΔrfaP to ΔrfaC, the strains bearing shorter LPS (ΔrfaG and ΔrfaC respectively) are more resistant to the beads, indicating that LPS has a role in the interaction with the bioactive beads.

Interestingly, an opposite effect was observed when bacteria were exposed to the free LK 20-mer random sequence peptide mixture, where the WT strain exhibited the lowest sensitivity and the ΔrfaC mutant was the most sensitive (FIG. 15B). In the case of the free random sequence peptides mixture, the ΔrfaG mutant, which possesses shorter LPS than the WT's but longer than ΔrfaC's, had an intermediate sensitivity. The higher activity that the free random sequence peptides mixture exhibited against ΔrfaC may suggest that LPS is not an important factor in the interaction in this case, presumably due to the high flexibility of the free random sequence peptides which enables them to easily contact bacterial cells.

The fundamental differences between the free and immobilized peptides indicate that the influence of membrane properties on the interaction between bacteria and random sequence peptides mixture differs in both cases, suggesting that the immobilization changes the mechanism of action of the random sequence peptides.

### Example 12

### Sterilization of Liquids by the Bioactive Beads

The ability of the bioactive beads to sterilize liquids was next examined. For that aim, an antimicrobial column, 10 mm diameter and 40 mm length of active layer, was packed with a mixture of 5% LK 20-mer beads in sterile sand. Phosphate buffered saline (PBS) inoculated with ˜10⁴ CFU/ml E. coli was pumped through the column and the bacterial cell viability in the treated water was measured.

As shown in FIG. 16, a significant reduction in the bacterial load was observed, with the column maintaining effectiveness after 8 hours of flow. The activity of the bioactive beads was found to be maintained for weeks and even for months. These results indicated that the bioactive beads are useful for purification of liquids under flow, the antimicrobial activity is preserved for long periods of time, and therefore are useful for water purification.

### Example 13

### Antimicrobial Activity of Different Immobilized Peptides Against Different Microorganisms

The next experiment aimed at evaluating the antibacterial activity of different immobilized random sequence peptides against various bacterial strains.

For that aim, random sequence 20-mer peptides consisting of leucine and lysine (LK) at two different ratios of 1:1 or 3:7, random sequence peptides consisting of phenylalanine and lysine (FK) at a ratio of 1:1, and random sequence peptides consisting of tryptophan and lysine (WK) at a ratio of 1:1 were synthesized on polystyrene-PAM-glycine beads. The antibacterial activity of the immobilized peptides against E. coli, Klebsiella aerogenes, Enterococcus faecalis, Pseudomonas aeruginosa, and HPC was then evaluated.

TABLE 2
Microbial load after treatment with immobilized random
sequence peptides.

| | | Microbial Load after AMPB* | | | |
|---|---|---|---|---|---|
| | | treatment (CFU/ml) | | | |
| | | AMPB* (4 mg) | | | |
| Tested bacteria | Inlet load | LK 1:1 | LK 3:7 | FK 1:1 | WK 1:1 |
| E. coli 8739 | 8.00E+06 | 0 | 0 | 5.75E+06 | 1.53E+04 |
| Klebsiella | 1.63E+07 | 1.30E+07 | 0 | 0 | 9.75E+06 |
| aerogenes | | | | | |
| Enterococcus | 9.75E+06 | 1.63E+06 | 0 | 0 | 0 |
| faecalis | | | | | |
| Pseudomonas | 2.50E+06 | 0 | 0 | 0 | 0 |
| aeruginosa | | | | | |
| HPC | 1.20E+03 | 0 | 0 | 0 | 0 |

*AMPB denotes antimicrobial peptides immobilized to beads.

The results in Table 2 indicated that immobilized random sequence peptides consisting of leucine and lysine at a ratio of 3:7 exhibited a higher antibacterial activity against E. coli, Klebsiella aerogenes, Enterococcus faecalis, and Pseudomonas aeruginosa than that of immobilized random sequence peptides consisting of leucine and lysine at a ratio of 1:1, phenylalanine and lysine at a ratio of 1:1, and tryptophan and lysine at a ratio of 1:1. It is noted that evaluation of the antibacterial activity of free random sequence peptides consisting of leucine and lysine at various ratios showed that the free peptides with the ratio of leucine and lysine of 1:1 exhibited the highest antibacterial activity against four different bacteria strains, while the free peptides with the ratio of leucine and lysine of 3:7 exhibited a lower antibacterial activity (see Hayouka et al., 2013, ibid). These results indicate that the contribution of hydrophobic amino acids and cationic amino acids to the antimicrobial activity of the random sequence peptides is affected upon their immobilization to a hydrophobic or water-insoluble resin, and demonstrate that random sequence peptides immobilized to a hydrophobic resin require a higher amount of lysine than leucine in order to achieve high antibacterial activity.

The antiviral activity of the immobilized random-sequence peptides was then evaluated against bacteriophage MS2.

TABLE 3
Microbial load after treatment with immobilized random sequence
peptides.

| | | Microbial Load after AMPB treatment * | | | |
|---|---|---|---|---|---|
| | | AMPB (4 mg) | | | |
| Test method | Inlet load | LK1:1 | LK 3:7 | FK1:1 | WK1:1 |
| bacteriophage | 5.00E+03 | 2.80E+03 | 2.43E+03 | 1.19E+03 | 0 |
| MS2 | | | | | |

* AMPB denotes antimicrobial peptides immobilized to beads.

As seen in Table 3, while all the immobilized random sequence peptides exhibited antimicrobial activity against bacteriophage MS2, the random sequence peptides consisting of tryptophan and lysine at a ratio of 1:1 exhibited the highest activity. These results therefore indicate that the immobilized random sequence peptides are highly potent antimicrobial agents and that a combination of two or more of these immobilized random sequence peptides can be used as a highly potent means for eradicating viable microorganisms.

It will be appreciated by persons skilled in the art that the present invention is not limited by what has been particularly shown and described herein above. Rather the scope of the invention is defined by the claims that follow.

1.-70. (canceled)

71. An antimicrobial matrix comprising a water-insoluble polymer, a linker, and a mixture comprising a plurality of synthetic peptides attached to the water-insoluble polymer via the linker,
wherein the plurality of synthetic peptides comprises cationic amino acid residues, hydrophobic amino acid residues, or any combinations thereof,
wherein the ratio between the total amount of the cationic amino acid residues and the total amount of the hydrophobic amino acid residues within the plurality of synthetic peptides ranges from about 10:1 to about 1:10, and
wherein the cationic amino acid residues and the hydrophobic amino acid residues are organized in the plurality of synthetic peptides in random sequences.

72. The antimicrobial matrix according to claim 71, wherein the cationic amino acid residues are selected from the group consisting of lysine, arginine, histidine, di-amino butyric acid (Dab), ornithine, and any combinations thereof.

73. The antimicrobial matrix according to claim 71, wherein the hydrophobic amino acid residues are selected from the group consisting of leucine, phenylalanine, tryptophan, valine, alanine, isoleucine, glycine, tyrosine, and any combinations thereof.

74. The antimicrobial matrix according to claim 71, wherein the peptides comprise one species of cationic amino acid residues and one to three species of hydrophobic amino acid residues.

75. The antimicrobial matrix according to claim 74, wherein the peptides consist of lysine and leucine or lysine and phenylalanine or lysine and tryptophan.

76. The antimicrobial matrix according to claim 71, wherein the peptides consist of an identical number of amino acid residues ranging from 5 to 50 amino acid residues in length.

77. The antimicrobial matrix according to claim 75, wherein the peptides consist of lysine and leucine, wherein the ratio between the total amount of lysine and the total amount of leucine ranges from about 5:1 to about 1:1, and wherein the peptides consist of an identical number of amino acid residues ranging from 10 to 30 amino acid residues in length.

78. The antimicrobial matrix according to claim 71, wherein the linker forms a distance between the peptide and the water-insoluble polymer of about 5 Å to about 20 Å.

79. The antimicrobial matrix according to claim 71, wherein the peptide and the water-insoluble polymer each is independently bound to the linker via an acid stable covalent bond

80. The antimicrobial matrix according to claim 79, wherein the linker comprises at least two functional groups, each is independently selected from the group consisting of an amine group, a carboxylic acid group, a thiol group, a maleimide (MI) group, 6-aminohexanoic acid, an azide group, and an acetylene group.

81. The antimicrobial matrix according to claim 80, wherein the linker comprises a compound selected from the group consisting of 4-hydroxymethyl-phenylacetamidomethyl (PAM), polyethylene glycol, an amino acid, and any combinations thereof.

82. The antimicrobial matrix according to claim 71, wherein the water-insoluble polymer is selected from the group consisting of polystyrene, polyethylene, polycarbonate, polypropylene, polysulfone, polymethyl methacrylate, acrylonitrile, polyethylene terephthalate, polyamide, agarose, sepharose, acrylate, cellulose, cross-linked cellulose, cellulose acetate, nitrocellulose, polyvinylidene fluoride (PVDF), silica, glass, gold, and metals.

83. The antimicrobial matrix according to claim 71, wherein the water-insoluble polymer is present in a form selected from the group consisting of a bead, a sphere, a microparticle, a nanoparticle, a fiber, a mesh, a net, a web, a grid, a lattice, and any combinations thereof.

84. An antimicrobial matrix comprising a water-insoluble polymer and a mixture comprising a plurality of synthetic peptides attached thereto,
wherein the peptides consist of one species of cationic amino acid residues, one to three species of hydrophobic amino acid residues, or any combinations thereof,
wherein the ratio between the total amount of the one species of cationic amino acid residues and the total amount of the one to three species of hydrophobic amino acid residues ranges from about 5:1 to about 1:1, and
wherein said one species of cationic amino acid residues and said one to three species of the hydrophobic amino acid residues are organized in the plurality of peptides in random sequences.

85. The antimicrobial matrix according to claim 84, wherein the peptides consist of an identical number of amino acid residues ranging from 5 to 50 amino acid residues in length.

86. The antimicrobial matrix according to claim 84, further comprising a linker capable of linking the peptide and the water-insoluble polymer.

87. The antimicrobial matrix according to claim 84, wherein the water-insoluble polymer is selected from the group consisting of polystyrene, polyethylene, polycarbonate, polypropylene, polysulfone, polymethyl methacrylate, acrylonitrile, polyethylene terephthalate, polyamide, agarose, sepharose, acrylate, cellulose, cross-linked cellulose, cellulose acetate, nitrocellulose, polyvinylidene fluoride (PVDF), silica, glass, gold, and metals.

88. The antimicrobial matrix according to claim 84, wherein the water-insoluble polymer is present in a form selected from the group consisting of a bead, a sphere, a microparticle, a nanoparticle, a fiber, a mesh, a net, a web, a grid, a lattice, and any combination thereof.

89. An article adapted to eliminate or exterminate viable microorganisms from a sample, the article comprising the antimicrobial matrix according to claim 71.

90. An article adapted to eliminate or exterminate viable microorganisms from a sample, the article comprising the antimicrobial matrix according to claim 84.

91. A method for eliminating or exterminating viable microorganisms from a sample comprising contacting the sample with the antimicrobial matrix according to claim 71, thereby eliminating or exterminating the viable microorganisms.

92. A method for the purification or filtration of a sample from viable microorganisms comprising contacting the sample with the antimicrobial matrix according to claim 71, thereby purifying the sample from viable microorganisms.

93. A method for eliminating or exterminating viable microorganisms from a sample comprising contacting the sample with the antimicrobial matrix according to claim 84, thereby eliminating or exterminating the viable microorganisms.

94. A method for the purification or filtration of a sample from viable microorganisms comprising contacting the sample with the antimicrobial matrix according to claim 84, thereby purifying the sample from viable microorganisms.
